(19) United States
(12) Patent Application Publication
(10) Pub. No.: US 2026/0242717 A1
(43) Pub. Date: Aug. 20, 2026

(54) BIOMOLECULE PRODUCTION SYSTEM COMPRISING PRESSURE SENSORS FOR VOLUME MEASUREMENT

(71) Applicant: UNIVERCELLS TECHNOLOGIES SA, Nivelles (BE)

(72) Inventors: Laetitia De Viron, Nivelles (BE); Alexander Vanhaver, Nivelles (BE); Anne-Claire Douillard, Nivelles (BE); Jean-Christophe Drugmand, Nivelles (BE)

(21) Appl. No.: 18/880,138

(22) PCT Filed: Jun. 28, 2023
(86) PCT No.: PCT/EP2023/067679

§ 371 Date: Dec. 30, 2024

Related U.S. Application Data
(60) Provisional application No. 63/356,262, filed on Jun. 28, 2022.

(51) Int. Cl. C12M 1/34; C12M 1/00; C12M 1/12; C12M 1/36

(52) U.S. Cl. CPC C12M 41/40; C12M 25/18; C12M 41/44; C12M 41/48; C12M 47/10

(57) ABSTRACT

The current disclosure relates to a biomolecule production system for producing biomolecules, wherein said system comprises one or more vessels, such as a bioreactor vessel, a transfer vessel, a reagent vessel, a waste vessel and/or a collection vessel, and optionally a concentrator, wherein one or more of said vessels are equipped with at least a first and a second pressure sensor, said first pressure sensor measuring the hydrostatic pressure in the vessel and said second pressure sensor measuring the gas headspace pressure (such as air pressure) in said vessel.

### TECHNICAL FIELD

This document relates to the technical field of the production of biomolecules such as (recombinant) proteins, RNA, DNA, viral particles, viral vectors, viral vaccines, gene therapy products or antibodies and describes a system and method thereto.

### BACKGROUND

Due to the vast number of diseases caused by pathogenic bacteria and viruses, there remains a large demand in the field to produce biomolecules such as antibodies and viruses efficiently. Biomolecule production systems developed to efficiently produce said biomolecules are widely known and are for instance described in EP3688134. EP3688134 describes a bioreactor vessel for culturing cells, a concentrator and a collection vessel suited to receive outflow from said concentrator and recycling it back to said concentrator or to a downstream process, allowing to obtain a heavily concentrated biomolecule product.

During production of biomolecules, accurate determination of the volume, and hence the level of fluid, inside one or more of the vessels (such as the bioreactor or collection vessel) of the biomolecule production system is necessary. Calculating the level of fluid inside such a vessel by determining the weight is not optimal, as a weighing unit is often difficult to integrate in the biomolecule production system. For instance, the installation of load cells or a scale below a bioreactor vessel is not only expensive, but also difficult to integrate because the bioreactor is often connected to additional elements, for instance placed on a heating element and/or a magnetic driver.

Often level sensors using capacitive technology are used determine the level of fluid in a vessel. However, the foaming tendency of the cultivation media leads to foam inside the vessel and level sensors using capacitive technology do not allow to register the difference between foam and liquid, resulting in measurement errors. Likewise, liquid adhering to the wall, could interfere with the accurate liquid level determination when using level sensors with capacitive technology. In addition, the placement of such capacitive level sensors is often complex because their positioning requires a flat surface in contact with the fluid.

Furthermore, during production of the biomolecules it is important that the vessels remain sterile and determination of the volume inside said vessels should be done in an aseptic manner.

Accordingly, a need is identified for a manner in which to assess the level of fluid, in a less complicated, less expensive and more accurate manner, while maintaining aseptic conditions so as to protect against contamination (both internal to the collection vessel and external to it).

### SUMMARY

The present disclosure serves to provide a solution to one or more of above-mentioned disadvantages. To this end, the present disclosure relates to a biomolecule production system according to claim 1. More in particular, the disclosure provides a biomolecule production system for producing biomolecules, wherein said system comprises one or more vessels, such as a bioreactor vessel, a transfer vessel, a reagent vessel, a waste vessel and/or a collection vessel, and optionally a concentrator, wherein one or more of said vessels are equipped with at least a first and a second pressure sensor, said first pressure sensor measuring the hydrostatic pressure in the vessel and said second pressure sensor measuring the gas headspace pressure (such as air pressure) in said vessel.

Preferred embodiments of the biomolecule production system are shown in any of the claims 2 to 24. By providing one or more vessels comprising at least a first and second pressure sensor, the volume and/or the weight of the liquid in said vessel can be easily and accurately determined.

In a second aspect, the present disclosure relates to a biomolecule production system according to claim 25. More in particular, the disclosure provides a biomolecule production system for producing biomolecules, wherein said system comprises one or more single-use vessels, wherein at least one of the one or more single-use vessels is equipped with at least a first and a second pressure sensor, said first pressure sensor measuring the hydrostatic pressure in the vessel and said second pressure sensor measuring the gas headspace pressure (such as air pressure) in said vessel.

In a further aspect, the invention relates to a vessel comprising at least a first and second pressure sensor wherein the first and second pressure sensors are adapted for measuring an amount of liquid in the vessel.

In a further aspect, the invention relates to a vessel comprising at least a first and second pressure sensor wherein the first and second pressure sensors are adapted for measuring an amount of liquid in the vessel and the vessel comprises a liquid.

In a further aspect, the invention relates to a method for measuring the volume of liquid in a vessel, wherein said vessel is equipped with at least a first and second pressure sensor, said first pressure sensor measuring the hydrostatic pressure in the vessel and said second pressure sensor measuring the gas headspace pressure in said vessel and wherein the volume of liquid in said vessel is calculated based on said pressure measurements.

In a further aspect, the present disclosure relates to a method for producing a biomolecule according to claim 27. More in particular, the disclosure provides a method for producing a biomolecule by means of aforementioned system.

In a further aspect, the present disclosure relates to a method for producing a biomolecule according to claim 28. More in particular, the disclosure provides a method for producing a biomolecule, such as a protein, a virus or viral particle, or gene therapy product, comprising the steps of providing a biomolecule production system comprising a bioreactor vessel, a collection vessel, a concentrator and a waste vessel, collecting the harvest from said bioreactor vessel in a collection vessel and further concentrating said harvest by means of a concentrator, wherein one or more of said vessels are equipped with at least a first and second pressure sensor, said first pressure sensor measuring the hydrostatic pressure in the vessel and said second pressure sensor measuring the gas headspace pressure (such as the air pressure) in said vessel and wherein the volume and/or weight of liquid in said vessel is calculated based on said pressure measurements.

In a further aspect, the present disclosure relates to a use according to claim 37. More in particular, the disclosure relates to the use of aforementioned system for the production of biomolecules, such as proteins, viruses and/or viral vaccines.

In a last aspect, the present disclosure relates to a method for determining the total liquid volume in a vessel according to claim 40. More in particular, the disclosure provides a method for determining the total liquid volume in a vessel of a biomolecule production system by means of a process controller, said vessel comprising at least a first and a second pressure sensor coupled to said process controller, wherein said total liquid volume consists of a first volume of liquid below the first pressure sensor and a second volume of liquid above the first pressure sensor, said total liquid volume is determined by: calculating the first volume of liquid and adding this to the second volume of liquid, said second volume of liquid being determined by measuring the hydrostatic pressure by means of the first pressure sensor, measuring the gas headspace pressure (such as the air pressure) by means of the second pressure sensor, thereby determining the differential pressure and calculating the volume of fluid above the first pressure sensor, wherein the differential pressure is comprised in a range between 0 to 200 mbar.

### DESCRIPTION OF FIGURES

The following description of the figures of specific embodiments of the disclosure is merely exemplary in nature and is not intended to limit the present teachings, their application or uses. Throughout the drawings, corresponding reference numerals indicate like or corresponding parts and features.

FIGS. 1A-C illustrate an embodiment of the collection vessel of the system according to the current disclosure.

FIG. 2 illustrates a front view of an embodiment of the system according to the current disclosure.

FIG. 3 shows a top view of the system of an embodiment according to the current disclosure.

FIG. 4A shows a front view of the system of an embodiment according to the current disclosure.

FIG. 4B shows a back view and a front view of the system of an embodiment according to the current disclosure.

FIG. 5 illustrates a detail of the front view of the system including the front windows of an embodiment according to the current disclosure.

FIGS. 6A and 6B illustrate an embodiment of the system according to the current disclosure, including the collection vessel and TFF.

FIG. 7 is a perspective view of a first embodiment of a bioreactor according to the current disclosure.

FIG. 8 is a perspective view of a bioreactor of FIG. 7, including several enlarged views.

FIGS. 9A and 9B illustrate a matrix material for use in forming a structured fixed bed for culturing cells in any of the disclosed bioreactors.

FIG. 10 illustrates a modular version of an embodiment of a bioreactor according to the current disclosure.

FIG. 11 is a cross-sectional view of an embodiment of a bioreactor according to the current disclosure.

FIG. 12 is a cross-sectional view of a base portion of the bioreactor of FIG. 11.

FIG. 13 is a partially cutaway top view of an intermediate part of the bioreactor of FIG. 11.

FIG. 14 is a partially cutaway top view of an intermediate part of the bioreactor of FIG. 11.

FIGS. 15, 15A and 15B are various view of an embodiment of a bioreactor according to the current disclosure.

FIG. 16 is a cross-sectional view of the bioreactor of FIG. 15.

FIG. 17 is a cross-sectional view of the bioreactor of FIG. 15.

FIG. 18 illustrates a process flow in an embodiment of the system according to the current disclosure.

FIG. 19 shows an embodiment of the system according to the current disclosure.

FIG. 20 shows an embodiment of the system according to the current disclosure depicting conductors for detecting the presence of foam in a vessel.

FIG. 21 shows an embodiment of the system according to the current disclosure depicting a bioreactor vessel and associated pressure sensors for determining the volume of liquid in said vessel.

FIG. 22 shows a schematic overview of a system for producing biomolecules according to an embodiment of the disclosure.

FIG. 23 shows a schematic representation of a cycle which allows to maximize the yield of target biomolecules in a biomolecule production system according to an embodiment of the disclosure, wherein either the liquid is simply recirculated through the concentrator or wherein the liquid is concentrated depending on the volume (or the weight) in the collection vessel as determined by means of the pressure sensors.

FIG. 24A shows a schematic representation of the liquid flow in a biomolecule production system according to an embodiment of the disclosure wherein the liquid is automatically recirculated through the concentrator (and no concentration of the bioharvest occurs) based on the volume (or the weight) in the collection vessel as determined by means of the pressure sensors.

FIG. 24B shows a schematic representation of the liquid flow in a biomolecule production system according to an embodiment of the disclosure wherein the liquid is automatically concentrated based on the volume (or the weight) in the collection vessel as determined by means of the pressure sensors.

FIG. 25A shows a schematic representation of the liquid flow in a biomolecule production system according to an embodiment of the disclosure wherein the feeding pump is automatically stopped based on the volume (or the weight) in the collection vessel as determined by means of the pressure sensors in order to prevent overfilling of the collection vessel.

FIG. 25B shows a schematic representation of the liquid flow in a biomolecule production system according to an embodiment of the disclosure wherein the draining of the collection vessel is automatically stopped based on the volume (or the weight) in the collection vessel as determined by means of the pressure sensors.

FIGS. 26A-B show an embodiment of the system according to the current disclosure depicting a bioreactor vessel, which can be provided with pressure sensors for determining the volume of liquid in said vessel.

### DETAILED DESCRIPTION

The present disclosure concerns a system and a method for the production of biomolecules such as proteins, RNA, DNA, viral particles, viral vectors, viral vaccines gene therapy products or antibodies. The present disclosure further relates to a method for determining the total liquid volume in a vessel.

Unless otherwise defined, all terms used in this disclosure, including technical and scientific terms, have the meaning as commonly understood by one of ordinary skill in the art to which this disclosure belongs. By means of further guidance, term definitions are included to better appreciate the teaching of the present disclosure.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in this disclosure. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the disclosure described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Reference throughout this specification to “one embodiment” or “an embodiment” means that a particular feature, structure or characteristic described in connection with the embodiment is included in at least one embodiment of the disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure, and form different embodiments, as would be understood by those in the art. For example, in the following claims, any of the claimed embodiments can be used in any combination.

“Biomolecule” refers to any biological material of interest that is produced in a bioreactor. Biomolecules include, for example, viruses, virus-like particles, viral products, gene therapy products, viral vectors, DNA, RNA, proteins such as antibodies, carbohydrates, lipids, nucleic acids, metabolites and peptides.

“Gene therapy product” refers to a therapeutic product comprising nucleic acids to treat or prevent a disease or disorder, such as a genetic disease or disorder.

“Viral gene therapy product” refers to a viral product where a part of the genetic material of the virus is substituted with therapeutic nucleic acids and where the virus is implemented to introduce the therapeutic nucleic acids into the cells of the patient. A number of viruses have been used for human gene therapy, including retroviruses, adenoviruses, herpes simplex, vaccinia, and adeno-associated virus.

“Antibody” refers to any immunoglobulin molecule, antigen-binding immunoglobulin fragment or immunoglobulin fusion protein, monoclonal or polyclonal, derived from human or other animal cell lines, including natural or genetically modified forms such as humanized, human, chimeric, synthetic, recombinant, hybrid, mutated, grafted, and in vitro generated antibodies. Commonly known natural immunoglobulin antibodies include IgA (dimeric), IgG, IgE, IgG and IgM (pentameric).

“Virus” or “virion” refers to an ultramicroscopic (roughly 20 to 300 nm in diameter), infectious agent that replicates only within the cells of living hosts, mainly bacteria, plants, and animals: composed of an RNA or DNA core, a protein coat, and, in more complex types, a surrounding envelope.

The biomolecule production system of the current invention can comprise one or more “vessels”. “Vessel” as used herein refers to a hollow container, especially one used to hold liquid. Examples of such a vessel include: a bioreactor vessel, a transfer vessel, a reagent vessel, a waste vessel and/or a collection vessel.

“Gas headspace pressure” as used herein refers to the pressure conferred by the gas present in the upper compartment of the vessel which is not filled with liquid. Said gas can be for instance air or a mixture of gases such as O₂, N₂ and/or CO₂. Air is a mixture of several gases, where the two most dominant components in dry air are 21 vol % oxygen and 78 vol % nitrogen.

“Bioreactor” and “bioreactor vessel” are used as synonyms and refer to any device or system that supports a biologically active environment inside a vessel, for example for cultivation of cells or organisms for biomass expansion and/or production of a biological product or biomolecule. This would include roller bottles, shaked flask, flatware flasks, stirred tank suspension bioreactors, high cell density structured or unstructured fixed-bed bioreactors, packed-bed bioreactors, microcarrier bioreactors, etc.

“Purification” refers to the substantial reduction of the concentration of one or more target impurities or contaminants relative to the concentration of a target biomolecule.

“Tangential flow filtration (TFF)” refers to a method of membrane filtration in which fluid is forced through a space bounded by one or more porous membranes, where molecules small enough to pass through the pores are eliminated in the filtrate or “permeate”, and molecules large enough to be rejected by the pores remain in the “retentate”. The name tangential flow particularly refers to the fact that the direction of fluid flow is roughly parallel to the membrane, as opposed to so-called dead-end filtration where flow is roughly perpendicular to the membrane.

“Cell culture harvest”, “culture harvest”, “bioharvest” and “(biomolecule or bioreactor) harvest” are used as synonyms and refer to the unclarified or clarified cell culture obtained from culturing cells in a bioreactor. The cultured cells or the grown cells also are referred to as host cells. In the current disclosure a harvest from a bioreactor vessel can for instance be further concentrated by means of a concentrator and collected in a collection vessel.

A “collection vessel” as described herein refers to a vessel that receives liquid output from the bioreactor vessel. In an embodiment, the collection vessel is connected to a concentrator, allowing re-circulation of retentate back and forth from the collection vessel to the concentrator and finally allowing harvesting of a concentrated cell culture harvest in the collection vessel.

A “waste vessel” as described herein refers to a vessel where undesired material that is produced in the system or by-products of the process can be temporarily stored.

A “reagent vessel” as described herein refers to a vessel comprising a reagent (such as a buffer, base, a transfection reagent, . . . ) to be used during the biomolecule production process.

A “transfer vessel” as described herein refers to a vessel where material (for instance material that is produced in the system or by-products of the process) can be temporarily stored. A transfer vessel can for instance temporarily store liquid and/or biomolecules produced in the system of the current invention, for instance while waiting for the next part of the process to start, to handle variable flowrates between two parts of the process (thereby functioning as a “buffer vessel”) or to remove gas bubbles before further processing of the liquid and/or biomolecules.”

“Serial, in-line” means that devices or units are connected such that the outflow of one unit or device is directly fed into a subsequent unit or device, without intermediate storage.

“Single-use” as described herein refers to a product or item which is designed to be used once and then disposed of or destroyed, for instance a single-use vessel.

As used herein, “docking” means to make a stable connection between two elements, whereby the elements can for instance comprise either a receiving portion or a connecting portion. In this disclosure, docking can for instance occur between the bioreactor cabinet and the system for production of biomolecules or between the bioreactor itself and the bioreactor cabinet.

In a first aspect, the current disclosure provides a biomolecule production system for producing biomolecules, wherein said system comprises one or more vessels, such as a bioreactor vessel, a transfer vessel, a reagent vessel, a waste vessel and/or a collection vessel, and optionally a concentrator, wherein one or more of said vessels are equipped with at least a first and a second pressure sensor, said first pressure sensor measuring the hydrostatic pressure in the vessel and said second pressure sensor measuring the gas headspace (for instance air) pressure in said vessel.

In an embodiment, said system comprises a bioreactor vessel and optionally a concentrator and a collection vessel, wherein one or more of said vessels are equipped with at least a first and a second pressure sensor, said first pressure sensor measuring the hydrostatic pressure in the vessel and said second pressure sensor measuring the gas headspace pressure (such as air pressure) in said vessel.

In an embodiment, the invention relates to a biomolecule production system for producing biomolecules, wherein said system comprises a bioreactor vessel and optionally a concentrator and a collection vessel, wherein one or more of said vessels are equipped with at least a first and a second pressure sensor for determining the volume of liquid in said vessel, said first pressure sensor measuring the hydrostatic pressure in the vessel and said second pressure sensor measuring the air pressure in said vessel.

In an embodiment, the biomolecule production system comprises a bioreactor vessel equipped with at least a first and second pressure sensor. In an embodiment, the biomolecule production system comprises a collection vessel, wherein the collection vessel is equipped with at least a first and second pressure sensor. In an embodiment, the biomolecule production system comprises a transfer vessel, wherein the transfer vessel is equipped with at least a first and second pressure sensor. In an embodiment, the biomolecule production system comprises a reagent vessel, wherein the reagent vessel is equipped with at least a first and second pressure sensor. In an embodiment, the biomolecule production system comprises a waste vessel, wherein the waste vessel is equipped with at least a first and second pressure sensor. In an embodiment, the biomolecule production system comprises a bioreactor vessel, a transfer vessel, a reagent vessel, a waste vessel and/or a collection vessel, wherein one or more of said vessels are equipped with at least a first and second pressure sensor. In an embodiment, the biomolecule production system further comprises a concentrator.

In an embodiment, in the biomolecule production system of the current disclosure, liquid output from the bioreactor vessel will be transferred to a collection vessel and this collection vessel is connected to a concentrator (for instance a TFF). The retentate of the concentrator will subsequently be brought back to the collection vessel, whereas liquid waste will be discarded (preferably to a waste bottle). Due to the re-circulation of retentate back and forth from the collection vessel to the concentrator, a heavily concentrated biomolecule product will be obtained. Finally, the recirculated output of the concentrator is harvested in said collection vessel thereby obtaining a concentrated cell culture harvest. The presence of a collection vessel offers the advantage that the bioreactor can be rinsed to harvest remaining liquid, while the volume of this rinsing liquid can still be reduced by the concentrator prior to further downstream processing.

Determination of the liquid level in the vessels of the biomolecule production system is important, for instance to characterize the content inside said vessels, to prevent overfilling of said vessels or to maintain a constant volume in said vessels. This is especially true for systems operating with a perfusion bioreactor (where culture medium is continuously exchanged: fresh medium replenishes nutrients, while cellular by-products waste and medium depleted of nutrients are removed) and where a collection vessel and concentrator are present.

In an embodiment, the pressure sensors are used to determine the liquid level inside the vessel which can be used to characterize the content inside said vessel. For instance, the liquid level inside the vessel can be used to characterize the concentration of target biomolecules inside the collection vessel in the final cell culture harvest after concentration by the concentrator and to determine when concentration of the harvest is sufficient and can be halted.

In an embodiment, determination of the liquid level in the vessels of the biomolecule production system allows to prevent overfilling in said vessels.

Likewise, in order to maintain a constant concentration level in the collection vessel throughout diafiltration and/or clarification, the addition of the buffer is metered by adjusting the flow rate of the buffer pump based on the weight (and hence level) measurement as determined by means of the pressure sensors. As such, in an embodiment, determination of the liquid level in the vessels of the biomolecule production system allows to maintain a constant volume in said vessels, for instance during perfusion, diafiltration or clarification.

However, measurement of the volume inside such a vessel using a scale or a load-cell is expensive and difficult to implement because of the integration of the vessel in the biomolecule system. As such, often level sensors using capacitive technology are used to determine the level of fluid in such a vessel. However, when foam is present in the vessel, the capacitive technology does not allow to register the difference between foam and liquid, resulting in measurement errors. Likewise, liquid adhering to the wall, could interfere with the accurate liquid level determination when using level sensors with capacitive technology.

As such, one or more of the vessels of the biomolecule production system according to the present disclosure are equipped with at least a first and a second pressure sensor which allow to determine the volume and/or the weight of the liquid in said vessel. The ability to determine the liquid level from the pressure in the vessel is based on Pascal's Principle. Pascal's Principle states that in a static environment, the depth of a liquid generates a force that is directly proportional to the height of the liquid. This principle can be represented by Equation 1 below, where ΔP is the hydrostatic pressure, p is the volumic mass, g is the acceleration due to gravity, and Δh is the height of the liquid.

$$Δ P = ρ g ( Δ h ) \qquad Equation 1$$

This equation can be used when the fluid density (p) and the geometry of the vessel are known. In an embodiment, the fluid inside the vessel is a cell culture medium with a fluid density close to that of water (1 g/m³). Using the geometry of the vessel, once the height is known, a simple calculation involving the cross-sectional area in the case of a vessel with unchanged cross-section (such as cube, a vertical cylinder or parallelepiped) can be used to determine the volume of liquid present. Obviously, in case of a more complex geometry, a more complex calculation should be done.

Similarly, when the volumic mass of the fluid is unknown, the hydrostatic pressure ΔP allows to determine the weight of the liquid inside the vessel according to Equation 2 (see below), where S is surface area. The weight inside the vessel can be used as a proxy to the liquid volume inside the vessel.

$$m = Δ P \times S / g \qquad Equation 2$$

More in detail, by knowing that: V=Δh×S and m=V. ρ, the equation becomes: m=ΔP×S/g.

In order to accurately determine the liquid level and/or the weight in the vessel via a pressure measurement, there are two important requirements: locational accuracy (the physical location of the sensors), and the accuracy and resolution of the pressure reading in low pressure ranges.

The first pressure sensor measures the hydrostatic pressure in the vessel. It is important that said first pressure sensor is located as close as possible to the bottom wall of the vessel. In an embodiment, said first pressure sensor is located in or near the lower half of the vessel, preferably at a height that is equal or smaller than ¼^{th} of the total length of said vessel, which height is measured from the bottom wall of said vessel. In an embodiment, said first pressure sensor is located at a height that equals ¼^{th} of the total length of said vessel. In an embodiment, said first pressure sensor is located at a height that is smaller than ¼^{th} of the total length of said vessel, preferably smaller than ⅕^{th}, more preferably smaller than ⅙^{th}, more preferably smaller than 1/7^{th}, more preferably smaller than ⅛^{th}, more preferably smaller than 1/9^{th}, more preferably smaller than 1/10^{th} of the total length of said vessel. In an embodiment, said first pressure sensor is positioned on the inside of the vessel. In another embodiment, said first pressure sensor is positioned on the outside of the vessel. In an embodiment, said first pressure sensor is connected to the wall of the vessel. In an embodiment, said first pressure sensor is connected to the inside wall of the vessel. In another embodiment, said first pressure sensor is connected to the outside wall of the vessel. In an embodiment, said first pressure sensor is indirectly connected to the vessel and its content, for instance by means of tubing or the like.

The second pressure sensor measures the gas headspace pressure (such as the air pressure) in said vessel. It is important that said second pressure sensor is located as high as possible with regard to the top wall of the vessel, to ensure that the correct gas headspace pressure is measured, even when the vessel is almost completely filled with liquid. In an embodiment, said second pressure sensor is located in or near the upper half of the vessel, preferably at a height that is equal or greater than ¾^{th} of the total length of said vessel, which height is measured from the bottom wall of said vessel. In an embodiment, said second pressure sensor is located at a height that equals ¾^{th} of the total length of said vessel. In an embodiment, said second pressure sensor is located at a height that is greater than ¾^{th} of the total length of said vessel, preferably greater than ⅘^{th}, more preferably greater than ⅚^{th}, more preferably greater than 6/7^{th}, more preferably greater than ⅞^{th}, more preferably greater than 8/9^{th}, more preferably greater than 9/10^{th} of the total length of said vessel. In an embodiment, said second pressure sensor is positioned on the inside of the vessel. In another embodiment, said second pressure sensor is positioned on the outside of the vessel. In an embodiment, said second pressure sensor is connected to the wall of the vessel. In an embodiment, said second pressure sensor is connected to the inside wall of the vessel. In another embodiment, said second pressure sensor is connected to the outside wall of the vessel. In an embodiment, said second pressure sensor is indirectly connected to the vessel and its content, for instance by means of tubing or the like.

In an embodiment, said one or more vessels are equipped with additional pressure sensors to determine the volume and/or weight of liquid in said vessel more accurately. Measuring the pressure at multiple points allows in certain instances to more accurately determine the volume and/or weight of liquid present in the vessel. In an embodiment, said one or more vessels are equipped with 2, 3, 4, 5, 6, 7, 8, 9 or 10 pressure sensors to determine the volume and/or weight of liquid in said vessel.

A water column of 1 inch correlates to only 0.036 psi (0.0025 bar), therefore, even minimal error can significantly impact the reading. As such, it is important that the pressure sensors display a high accuracy and resolution in low pressure ranges.

Said pressure sensors can be any accurate pressure sensor known from the state of the art. In an embodiment, said pressure sensors are able to measure pressures up to 75 psi (5.2 bar). Said pressure sensors can be made from any material known from the state of the art. In an embodiment, said pressure sensors are made from caustic resistant polysulfone, to withstand sanitization processes. In an embodiment, said pressure sensors are made from polycarbonate. In an embodiment, the sensors can be electrically connected. In another embodiment, the sensors can be wireless.

With the ongoing growth of the biopharmaceutical, vaccine, and cell and gene therapy markets, and the increasing demand on existing sterilization facilities for materials required in manufacturing processes, gamma irradiation or X-ray irradiation for sterilization has caught the eye of the industry or any other sterilization method. In an embodiment, said pressure sensors are compatible with gamma irradiation. In an embodiment, said pressure sensors are compatible with X-ray irradiation for sterilization.

Within the pharmaceutical industry there are strict requirements to maintain sterility during production. As such, it is highly important that sterile conditions in the vessels are maintained and that the connection between the vessel and the pressure sensors occurs under sterile conditions. As such, in a preferred embodiment, the pressure sensors are integrated in the biomolecule production system in an aseptic manner. The pressure sensors could be immediately placed inside the vessel, however this set-up increases the risk of contamination inside the vessel. In an embodiment, the pressure sensors connect to the vessel via a custom port plate welded into the vessel.

In another embodiment, said pressure sensors are removably connected to said vessel in an aseptic manner. This increases flexibility and for instance allows to replace the pressure sensors without replacing the vessel.

In an embodiment, said pressure sensors are removably connected to said vessel by means of one or more clamps, flanges, caps and/or gaskets in an aseptic manner. In an embodiment, said one or more clamps are screw types. In an embodiment, said one or more gaskets are tri-clamp gaskets. Tri-clamp gaskets are mainly used in the food, dairy, beverage, biotech and pharmaceutical industries for sealing clamp connections in sanitary pipes. In an embodiment, said one or more clamps are made from a metal. In another embodiment, said one or more clamps are non-metallic. In an embodiment, said one or more clamps are made from a plastic, such as nylon—66. In an embodiment, said one or more clamps, flanges, caps and/or gaskets are compatible with gamma irradiation and/or X-ray irradiation for sterilization. In an embodiment, said one or more clamps, flanges, caps and/or gaskets are compatible for single-use applications.

In an embodiment, said vessel equipped with at least a first and a second pressure sensor is further equipped with a drain line, said drain line comprising the first pressure sensor. As discussed above, the positioning of the first pressure sensor is important and should be as close as possible to the bottom wall of the vessel. As such, it is important that the drain line (and hence the first pressure sensor) is positioned as close as possible to the bottom wall of the vessel. In an embodiment, said drain line is located in or near the lower half of the vessel, preferably at a height that is equal or smaller than ¼^{th} of the total length of said vessel, which height is measured from the bottom wall of said vessel. In an embodiment, said drain line is located at a height that equals ¼^{th} of the total length of said vessel. In an embodiment, said drain line is located at a height that is smaller than ¼^{th} of the total length of said vessel, preferably smaller than ⅕^{th}, more preferably smaller than ⅙^{th}, more preferably smaller than 1/7^{th}, more preferably smaller than ⅛^{th}, more preferably smaller than 1/9^{th}, more preferably smaller than 1/10^{th} of the total length of said vessel.

Said vessel, for instance said collection vessel, can be any collection vessel known from the state of the art. In an embodiment, said (collection) vessel is made from plastic, such as polypropylene (PP) or polyester (PES). In an embodiment, said (collection vessel) is made from polyethylene terephthalate (PET).

In an embodiment, the wall of said vessel equipped with at least a first and a second pressure sensor (for instance said collection vessel) has a thickness of at least 0.1 mm, more preferably at least 0.2 mm, more preferably at least 0.5 mm, more preferably at least 1 mm, more preferably at least 2 mm, more preferably at least 3 mm, more preferably at least 4 mm, more preferably at least 5 mm, more preferably at least 6 mm, more preferably at least 7 mm, more preferably at least 8 mm, more preferably at least 9 mm, such as 10 mm. In a preferred embodiment, the wall of said collection vessel has a thickness between 1 and 20 mm, more preferably between 5 and 15 mm, such as 10 mm. Such a wall thickness is necessary to obtain sufficient firmness and stability of the vessel. In the case of a vessel with unchanged cross-section (such as cube, a vertical cylinder or parallelepiped), in order to accurately measure the cross-sectional area and calculate the volume of liquid in the vessel, it is mandatory that the vessel is not flexible.

In a preferred embodiment, the collection vessel is connected to one or more inlet and/or outlet tubings. In an embodiment, the collection vessel is connected to one or more inlet and/or outlet gas lines. In an embodiment, said gas lines are protected by vents. In a preferred embodiment, the collection vessel comprises an inlet for small additions. In a preferred embodiment, the collection vessel comprises an inlet for gas addition (such as air, N₂, O₂, CO₂). In an embodiment, said CO₂ is used to maintain a stable pH inside the collection vessel (when culture media is based on a carbonate buffer). In an embodiment, the collection vessel comprises one or more outlets for gas. In a preferred embodiment, the collection vessel comprises an inlet for one or more buffers. In a preferred embodiment, the collection vessel comprises a conduit for connection to a bubble trap. The generation of foam during the course of a bioprocess remains a major technological challenge to be resolved. The foaming tendency of the cultivation media used in vessels induces various direct, that is microbial cells stripping and contamination, as well as indirect adverse effects, that is modification of the properties of the medium subsequent to the addition of chemical antifoam leading to toxic effects at the level of the microbial metabolism and fouling of the downstream processing equipment. In an embodiment, the system comprises a foam trap to remove foam from the system.

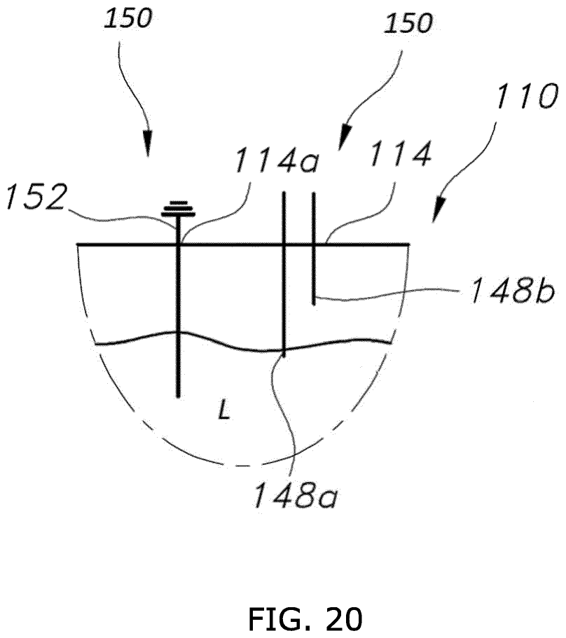

In an embodiment, one or more of the vessels are equipped with means for the detection of foam. Any sensor for the detection of foam known in the art can be used. In an embodiment, one or more of the vessels comprised in the system are formed of a material insulative to a liquid medium when present therein, the system further comprising one or more conductors. In an embodiment, one or more of said conductors comprises a conductive pin or wire connecting the liquid medium with an external structure. In an embodiment, said conductors may be used to detect the presence of foam as described in FIG. 20.

In an embodiment, the collection vessel comprises one or more handles for easy transport of the collection vessel. In an embodiment the collection vessel may be single-use, disposable and/or autoclavable. The shape of said vessel may be any kind of shape known to the skilled person and suited for its purpose.

In an embodiment, said biomolecule production system further comprises means for measuring the pH inside one or more of said vessels. In an embodiment, the pH sensor is multi-use. In an embodiment, the pH sensor is single-use. In an embodiment, the part of the pH sensor which is placed into the vessel (such as the probe) is single-use, whereas the part of the sensor not in contact with the vessel is multi-use (such as the transmitter of a pH sensor).

In an embodiment, said (collection) vessel is pressurized to prevent leakage. Said pressure can be in a range from 0 to 200 mbar, preferably in a range from 0 to 100 mbar, more preferably in a range from 0 to 50 mbar.

In an embodiment, said (collection) vessel is able to withstand a pressure of 200 mbar or more. This is necessary when gas injection in the vessel occurs.

The system's concentrator can be chosen from a number of devices known to the skilled person which are suited for reducing the volume of the liquid in which the target biomolecule resides. In some embodiments, the concentrator comprises one type of concentration device (e.g., tangential flow filter). In some embodiments, the concentrator comprises more than one type of concentration device (e.g., tangential flow filter and dead-end filter). Most of these devices are based on filtration and/or size exclusion chromatography. In one embodiment the concentrator is a filtration device, more preferably a micro-filtration device, or an ultra-filtration device or a combination of both micro- and ultra-filtration device. When the system is provided with an ultra-filtration device for reducing the volume of the liquid in which the target biomolecule resides, the membrane of the device is adapted as to allow flow through of water and low molecular weight solutes, which are in general referred to as the permeate, while macromolecules such as biomolecules are retained on the membrane in the retentate.

In an embodiment, said TFF is equipped with at least one hollow fiber having pores with a porosity sufficient to retain practically all of the target biomolecules, while permitting smaller contaminants such as growth medium and solutes to pass through the pores of the membrane. In contrast to dead-end filtration, in which the liquid is passed through a membrane or bed, and where the solids are trapped on the filter, tangential flow across the surface of the filter is allowed in the TFF device, rather than directly through the filter. Accordingly, formation of a filter cake in the TFF is avoided. In another embodiment, said TFF may be equipped with a cassette/cartridge allowing tangential flow filtration, said cassette/cartridge comprising ultrafiltration membranes allowing to retain practically all of the target biomolecules, while permitting smaller contaminants such as growth medium and solutes to pass through the pores of the membrane. In yet another embodiment, said TFF is a single pass tangential flow filtration (SP-TFF). This device is especially advantageous when purifying proteins such as antibodies. In some embodiments, the TFF comprises a membrane with an area of between 50 cm² and 20 m². In some embodiments, the TFF comprises a membrane with an area of between about 1000 cm² and 2000 cm², such as 1500 cm². The TFF may be reused, for one time use and/or disposable. In some embodiments, the TFF is plug and play.

As mentioned above, the system is provided with a retentate conduit mediating re-circulating of the retentate to an input of the bioreactor vessel or an input of the collection vessel. An additional advantage of implementing a TFF device as a concentrator in the system is that the TFF device is suited to be operated in a continuous perfusion process. This allows significant concentration of the culture volume.

In an embodiment, the system conduits are fitted with pumps and valves to provide directional liquid flow, to control differential pressure between different fragments of the system and to provide cross-flow of the liquid through the TFF concentrator. In an embodiment, the bioreactor and the collection vessel are connected by a conduit having a feeding pump, facilitating liquid transport from the bioreactor to said collection vessel. Alternatively, an additional conduit connected directly from the bioreactor to the concentrator could be present for transporting liquid from the bioreactor to the concentrator. In addition, the collection vessel and the concentrator are also connected by a conduit having pump which facilitates liquid transport from the collection vessel to the concentrator. The concentrator is able to enhance the amount of target biomolecule present in the liquid by enabling the reduction of the total liquid volume without reducing the amount of target molecule in the liquid. During such a concentration step, the permeate from the concentrator is transported towards a decontamination or waste vessel by means of a permeate conduit. Furthermore, in a preferred embodiment, the retentate line output which collects the concentrator output and which allows re-circulating of the retentate output to an input of a collection vessel is provided with a pressure control valve (PCV) which allows to maintain a specific transmembrane pressure (TMP) setpoint in the system.

As described above, an output conduit line from the concentrator to a decontamination vessel is provided to discard the permeate. As such, concentration of the liquid in the system can be obtained by the concentrator. However, when the output conduit line is closed, no permeate leaves the system, and the overall volume is simply recirculated through the concentrator back to the collection vessel. As described above, in a preferred embodiment, the liquid flow from the bioreactor to the collection vessel is controlled by means of a pump, which allows harvest feeding from the bioreactor to the collection vessel. When the output conduit line is closed, the volume of liquid in the collection vessel increases because of the harvest feeding from the bioreactor to the collection vessel.

Determination of the liquid level and/or liquid weight in the vessels of the biomolecule production system is important, for instance to characterize the content inside said vessels, or to prevent overfilling of said vessels or to maintain a constant volume in said vessels.

In an embodiment, the pressure sensors are used to determine the liquid level and/or liquid weight inside the vessel which can be used to characterize the content inside said vessel. For instance, the liquid level inside the vessel can be used to characterize the concentration of target biomolecules inside the collection vessel in the final cell culture harvest after concentration by the concentrator and to determine when concentration of the harvest is sufficient and can be halted. Determination of the liquid level (or weight) in the vessels of the biomolecule production system further allows to prevent overfilling in said vessels. Likewise, in order to maintain a constant concentration level in the collection vessel throughout diafiltration and/or clarification, the addition of the buffer is metered by adjusting the flow rate of the buffer pump (not shown) based on the level or weight measurement as determined by means of the pressure sensors.

The process flow in the system (from bioreactor to concentrator and/or collection vessel and between the concentrator and the collection vessel) is controlled by a process controller.

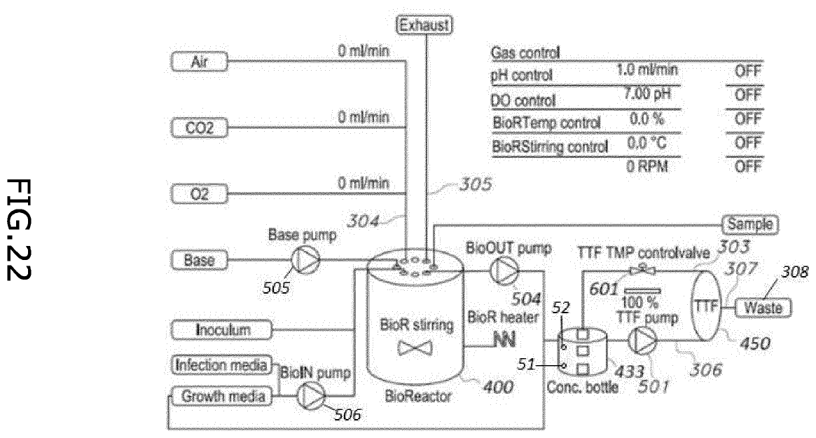

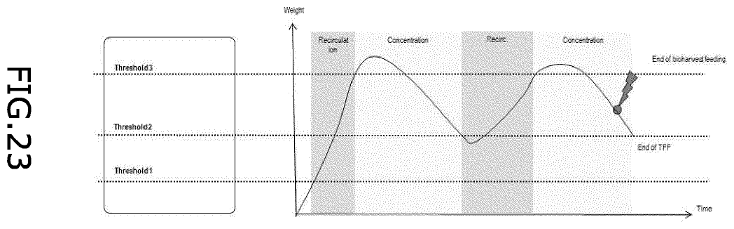

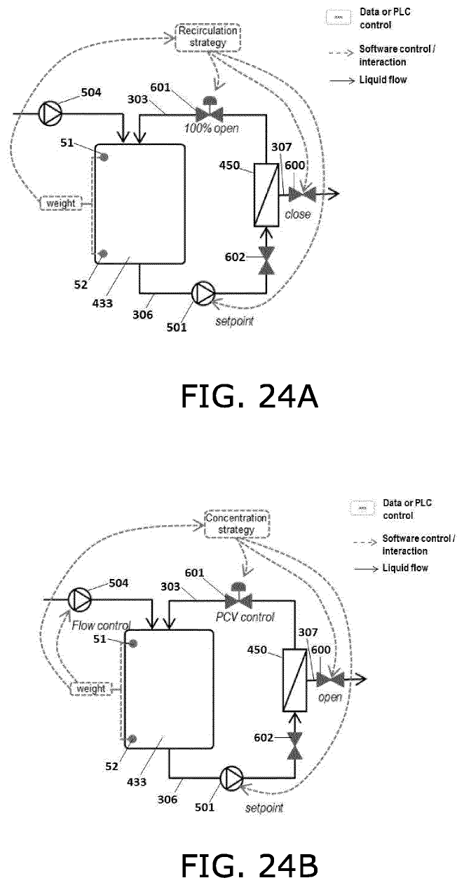

In an embodiment, the concentrator follows a cycle to maximize the yield of target biomolecules, wherein either the liquid is simply recirculated through the concentrator (referred to as “recirculation strategy”) or wherein the liquid is concentrated (referred to as “concentration strategy”) depending on the volume (or the weight) in the collection vessel (see FIGS. 23 and 24A-B) as determined by means of the pressure sensors. The chosen strategy is determined by certain threshold values (see FIG. 23). For instance, “Threshold 1” is the weight (or volume) to start the recirculation strategy (see FIG. 24A), during which the output conduit line (permeate line) is closed by means of valve, no permeate leaves the system and the volume and weight in the collection vessel increases by the harvest feeding from the bioreactor to the collection vessel. This weight increase can be determined by means of the first and second pressure sensor. At a certain point, “Threshold 3” is reached, indicating a high level in the collection vessel as measured by the pressure sensors, allowing the valve controlling the output conduit line (permeate line) to open and start the concentration strategy during which the permeate leaves the system and the retentate comprising the target biomolecule is recirculated to an input of the collection vessel (see FIG. 24B). “Threshold 2” is the final weight desired by the user at the end of the cycle. The end of harvest feeding automatically triggers the concentration until “Threshold 2”. These threshold values are configurable by the user.

During the recirculation strategy the permeate line is closed by a valve and simple recirculation through the TFF cartridge occurs while the PCV valve is 100% open. During the concentration strategy the permeate line is open, allowing the permeate to leave the system, while opening of the PCV valve is done to maintain a specific TMP setpoint in the system.

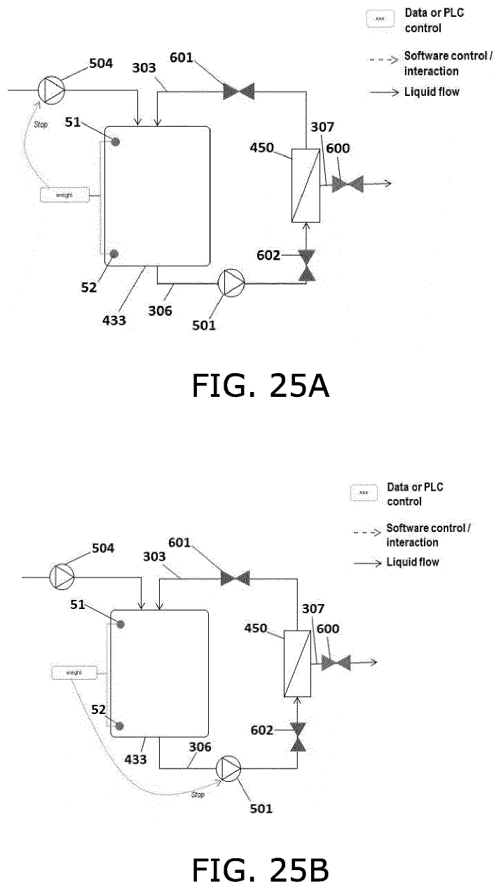

Furthermore, in an embodiment, based on the weight in the collection vessel as determined by means of the pressure sensors, the flow of the feeding pump during in-line perfusion and concentration is controlled to avoid overfilling and keep a constant weight (where the maximum level is for instance defined by threshold 3) (see FIGS. 23 and 25A). Similarly, besides monitoring and controlling the liquid level in the collection vessel during harvest feeding from the bioreactor, it is also important to determine and control the liquid level in the vessels of the biomolecule production system during diafiltration and clarification. In an embodiment, during constant-volume diafiltration and clarification, buffer is introduced into the collection vessel at the same rate that permeate is removed from the system. In order to keep the total volume of retentate constant (and to maintain a constant concentration level in the collection vessel) throughout the process, the addition of buffer is metered by adjusting the flow rate of the buffer pump based on the weight (and hence level) measurement as determined by means of the pressure sensors. Likewise, at the end of the harvest cycle the collection vessel needs to be drained. In an embodiment, based on the weight in the collection vessel (as determined by means of the pressure sensors) the end of the draining step of the collection vessel can be determined and the flow of the pump transporting liquid to the concentrator can be controlled to automatically stop the draining (and for instance prevent air or other gases from entering the filters) (see FIG. 25B). Due to the re-circulation of retentate back and forth from the collection vessel to the concentrator, a heavily concentrated biomolecule product will be obtained, which can be used for further downstream processing (such as chromatographic purification) or as source for trials such as e.g. clinical trials.

In an embodiment, said collection vessel is configured to be incorporated in said biomolecule production system.

In a further aspect, the invention relates to a vessel comprising at least a first and second pressure sensor wherein the first and second pressure sensors are adapted for measuring an amount of liquid in the vessel. As described above, said vessel can be for instance a bioreactor vessel, a transfer vessel, a reagent vessel, a waste vessel and/or a collection vessel.

In a further aspect, the invention relates to a vessel comprising at least a first and second pressure sensor wherein the first and second pressure sensors are adapted for measuring an amount of liquid in the vessel and the vessel comprises a liquid.

In an embodiment, the system further comprises a docking station, said docking station encompassing the bioreactor vessel. In an embodiment, the system comprises a docking station, said docking station encompassing the bioreactor vessel, the concentrator and the collection vessel. In a further embodiment, said collection vessel is positioned between said bioreactor vessel and said concentrator, wherein said collection vessel and concentrator are connected by a retentate conduit, allowing recirculating of liquid from an output of the concentrator to an input of said collection vessel. In an embodiment, said system further encompasses a process controller, integrated in said docking station, which is able to control the biomolecule process. In an embodiment, said docking station is formed by the casing of said process controller. In an embodiment, said docking station is sized to be operated within a laminar flow cabinet or biosafety cabinet, providing a benchtop system. Such a benchtop system may feature a touchscreen for quick-access function (e.g. pump priming, visual representation of live status and monitoring parameters) as well as docking slots for base and inoculum. The housing of the process controller can be made of any suitable material but is preferably manufactured out of stainless steel and is designed to enable user-friendly cleaning. In some embodiments the footprint occupied by the controller housing is less than about 5000 cm². Such a benchtop system integrates intensification technologies, thereby drastically reducing the size of each compartment and hence creating a low footprint production and purification system. The production and purification of the biomolecule can be performed as a continuous and automated process based on this system: from cell culture to final product purification minimizing human intervention. The process intensification and integration enable the containment of all compartments into an isolator ensuring the safety of process operators and the environment. The system has a small footprint. In some embodiments, the footprint of the system is less than about 50 m², 40 m², 30 m², 20 m², 10 m², 5 m², or less. In some embodiments, the footprint of the system is from about 5 m² to 10 m², 5 m² to 20 m², 5 to 30 m², 5 to 40 m², 5 to 50 m². In an example, the footprint is less than 10 m². For example, a 7 m² system can produce at least 0.5 million doses of a viral vaccine per batch, or about 107 doses per year. As a consequence, this autonomous process has a dramatic impact on the economics of biomolecule production by significantly reducing the cost of goods as well as capital expenditures. The system for producing biomolecules of the present disclosure allows down-scaling of the infrastructure required for biomolecule production on an industrial level, thereby also allowing to reduce the amount of consumables. The system reduces the amount of consumables used by greater than or equal to about 10%, 20%, 30%, 40%, 50%, 60%, 70%, 80%, 90%, or more. The system reduces the amount of consumables used from about 10% to 20%, 10% to 30%, 10% to 40%, 10% to 50%, 10% to 60%, 10% to 70%, 10% to 80%, 10% to 90%. The system further allows to purify a biomolecule in a safe, efficient and cost-effective manner. The system of the disclosure allows rapid production and purification of biomolecules such as recombinant proteins, viruses or viral products using significantly smaller equipment as compared to systems of the prior art. In addition, high yield of the biomolecule is obtained using the system, thereby reducing the costs of the final product. The recovery of the target biomolecule may be greater than or equal to 65%, 70%, 75%, 80%, 85%, 90%. This eventually results in a lower investment and production cost, which is a considerable advantage.

In an embodiment, said bioreactor vessel and optionally said collection vessel and said concentrator are comprised in a bioreactor chamber.

In a preferred embodiment, said bioreactor chamber comprises a wall or back sheet opposite to an operation area of said chamber, said wall or back sheet is provided with one or more instruments chosen from pumps, pipings, electrical sockets and/or manifolds needed for allowing functioning of said chamber. The collection vessel is suited to receive outflow from said concentrator and recycling it back to said concentrator or to a downstream process. In a preferred embodiment, said concentrator such as TFF as well as the collection vessel are connected to a back sheet of said bioreactor chamber. In a preferred embodiment the collection vessel with the TFF attached is located at the center of the bioreactor chamber behind the bioreactor vessel. The TFF is connected to the collection vessel with a support. The collection vessel—TFF—and TFF pump assembly is attached to the background metal sheet of the system. Said bioreactor vessel and said collection vessel are connected by a conduit, facilitating liquid transport from the bioreactor vessel through an inlet to said collection vessel. In one embodiment, the collection vessel is filled with the cell harvest of the bioreactor vessel using a pump. The homogeneity inside the collection vessel is guaranteed with the recirculation loop through the TFF with the TFF pump.

In an embodiment, said vessel equipped with at least a first and a second pressure sensor (for instance said collection vessel) has an internal volume of at most 100 liters, preferably at most 90 liters, preferably at most 80 liters, preferably at most 70 liters, preferably at most 60 liters, such as 50 liters. In an embodiment, said collection vessel has an internal volume between 1 and 100 liters, preferably between 10 and 80 liters, more preferably between 20 and 70 liters, more preferably between 30 and 60 liters, such as 50 liters. A collection vessel of such a size is easy to integrate in the biomolecule production system, for instance attached to the background metal sheet of the bioreactor chamber.

In an embodiment, said biomolecule production system further comprises at least one process chamber comprising one or more filtration or purification devices allowing the production of a biomolecule from a cell harvest.

The harvest can comprise of medium originating from the bioreactor vessel or can be cells or a lysate of the cells cultured in the bioreactor vessel. Said filtration or purification means can be a combination of one or more of clarification, floculation, precipitation of cell debris, lipids, host cell proteins, DNA, as well as ultrafiltration, tangential flow filtration aiming at concentrating the supernatant, or changing the chemical conditions (such as pH, conductivity, ionic strength). Said means can also be chromatographic means, in capture mode or in flow through mode; chromatography can be envisaged both in a packed mode, a monolith mode, a membrane-based mode or in a fluidized mode; should the chromatography be implemented in a fluidized mode, it can include the use of classical media separated by settling or centrifugation, or (para) magnetic media separated by an external magnetic field. It can be any combination of any of the means described previously. Such devices may include but are not limited to one or more chromatography column such as affinity chromatography, ionic exchange chromatography (e.g. anion or cation), hydrophobic interaction chromatography, size exclusion chromatography (SEC), immuno-affinity chromatography which is a column packed with an affinity resin, such as an anti-IgM resin, a Protein A, a Protein G, or an anti-IgG resin or any combination. Anion exchange exploits differences in charge between the different products contained in the harvested supernatant. The neutrally charged product passes over the anion exchange chromatography column cartridge without being retained, while charged impurities are retained. The size of the column may vary based on the type of protein being purified and/or the volume of the solution from which said protein is to be purified.

In an embodiment, the system is a mobile system, comprising wheels or tracks to allow transport.

In an embodiment, the biomolecule production system can comprise one or more process controllers. In an embodiment, one or more process controllers are configured to control the bioreactor vessel, the collection vessel and/or the concentrator of the biomolecule production system. In some embodiments, the process controller is configured to control operations of a biomolecule production system and can include a plurality of sensors, a local computer, a local server, a remote computer, a remote server, or a network.

In an embodiment, the process controller can be operational to control aspects of a product manufacturing process, and can be coupled to sensors disposed in the biomolecule production system, for example, to control the temperature, volume flow rate or gas flow rate in the bioreactor vessel of the biomolecule production system in real time.

In an embodiment, the process controller is coupled to said first and second pressure sensors disposed in one or more of the vessels of the biomolecule production system. In an embodiment, said process controller determines the total liquid volume in one or more of the vessels of the biomolecule production system by means of the measurements of said first and second pressure sensors comprised in one or more vessels of the biomolecule production system. In an embodiment, said process controller controls the liquid level of one or more of the vessels in the biomolecule production system based on the measurements of said first and second pressure sensors. In an embodiment, said process controller controls the liquid level in the bioreactor vessel by adjusting the flow rate of the liquid entering and/or exiting the bioreactor vessel based on the measurements of said first and second pressure sensors. In an embodiment, the sensors can be electrically connected to the process controller. In an embodiment, the sensors can be wirelessly connected to the process controller.

In an embodiment, the process controller is divided in two parts, namely a Programmable Logic Controller (PLC) and a Supervisory Control and Data Acquisition (SCADA). The PLC is the intelligence of the system and is connected to the sensors and the actuators. The PLC contains only data and no power. The SCADA is important for visualisation, data historian and audit trail. This SCADA system runs on a server that stores the data historians and supports the visualization. In an embodiment, information can also be visualized from a client tablet. In an embodiment, the client network can be connected directly to the server for remote access. In some embodiments, a process controller can include a Human-Machine Interface (HMI), such as a display, for example, a computer monitor, a smart phone app, a tablet app, or an analog display, that can be accessed by a user to determine the state of the system (based on the sensors comprised in the system) and to control the system by means of various actuators, such as pumps, valves, heaters and agitators. In some embodiments, the process controller can include an input, for example, a keyboard, a separated smart tablet, a key pad, a mouse, or a touch screen, to allow a user to enter control parameters for controlling the operation of the bioreactor vessel. In some embodiments, the process controller can control access to the biomolecule production system.

In an embodiment the system is equipped with control software. This software enables the gathering, transmission, processing and visualisation of parameter measurements in the system. In addition, the control software will be able to adjust these parameters. Parameters include but are not limited to pH, temperature, dissolved oxygen, volume, nutrients and pressure. In an embodiment, the control software is able to display alarm signals when the system does not operate appropriately. In an embodiment, it is possible to access the system remotely via connection to a network. In an embodiment the system is controlled by the user through a smart tablet connected to the control system.

In an embodiment, the biomolecule production system can include one or more additional sensors besides the pressure sensors, for example, a temperature sensor (e.g., a thermocouple), flow rate sensor, gas sensor or any other sensor. In some embodiments, the biomolecule production system disclosed herein can comprise and or contain sensors for monitoring different parameters. In an embodiment, the sensors can be electrically connected. In another embodiment, the sensors can be wireless. In another embodiment, the biomolecule production system comprises both electrically connected sensors and wireless sensors. In some embodiments, a sensor disclosed herein can be located in any compartment of the biomolecule production system disclosed herein. In some embodiments, sensors described herein can be a gas sensor (e.g. oxygen, nitrogen, or carbon dioxide), pH sensor, temperature sensor, cell density sensor, level sensor or dissolved oxygen (DO) sensor. In some embodiments, the sensors disclosed herein can measure amongst other things, biomass or cell density, the dissolved oxygen partial pressure, oxygen content, the pH value, the temperature, pressure, flow rate, certain concentrations of nutriments, such as lactate, ammonium, carbonates, glucose or any metabolic product or product to be metabolized which could for example reflect the cell density. In some embodiment, cell density (biomass density) can be determined by electrical impedance analysis or electrical impedance spectroscopy using an arrangement of measuring electrodes.

In some embodiments, a bioreactor vessel according to the disclosure can comprise sensors for measuring culture parameters. In some embodiments, a sensor disclosed herein can be in contact with culture medium in the bioreactor vessel. In some embodiments, culture parameters can comprise amongst other things, the dissolved oxygen partial pressure, the pH, the temperature, the optical density, certain concentrations of nutriments, such as lactate, ammonium, carbonates, glucose or any metabolic product or product to be metabolized which could for example reflect the cell density. In an embodiment, the part of the sensor which is placed into the bioreactor vessel (for instance a pH probe) is single-use, whereas the part of the sensor not in contact with the bioreactor vessel is multi-use (such as the transmitter of a pH sensor). In some embodiment, a bioreactor vessel disclosed herein can use regulation loops according to the disclosed parameters. In some embodiments, a regulation loop can for example, modulate the quantity of oxygen to be injected according to the value of the dissolved oxygen partial pressure present or the quantity of dissolved oxygen consumed by the cells; speed of circulation of the culture medium; inject CO₂ according to the pH value obtained by the sensors or any other type of regulation generally used in this type of culture. In some embodiments, cells can be exposed to dissolved oxygen concentrations of 300 mM or less (160 mmHg partial pressure), less than 200 mM, or between 20 and 150 mM. In some embodiments, cells can be exposed to about 0%, 1%, 5%, 10%, 20%, 30%, 40%, 50%, 60%, 70%, 78%, 80%, 90%, or 100% nitrogen and/or about 0%, 1%, 5%, 10%, 21%, 30%, 40%, 50%, 60%, 70%, 80%, 90% or 100% oxygen. In some embodiments, cells can be exposed to pure oxygen or an oxygen enriched atmosphere. In an embodiment, a sampling assembly can be connected to the bioreactor vessel lid.

The concentrator of the biomolecule production system is equipped with a retentate conduit suitable for collecting a retentate and facilitating re-circulating of the retentate to an input of said bioreactor vessel or to an input of said collection vessel. In an embodiment, the concentrator is controlled by one or more valves, such as pinch valves. Said bioreactor vessel and said concentrator are connected by a conduit facilitating liquid transport from said bioreactor vessel to said concentrator. Likewise, said collection vessel and said concentrator are connected by a conduit facilitating liquid transport from said collection vessel to said concentrator. In an embodiment, the liquid is pumped from the collection vessel to the concentrator by means of pump. In a further embodiment, the pump is a single-use pump. In a preferred embodiment, the pump is a single-use diaphragm pump. In an embodiment, the collection vessel comprises a conduit allowing the fluid to by-pass the concentrator. In an embodiment, said bioreactor vessel and said collection vessel are connected by a conduit, facilitating liquid transport from said bioreactor vessel to said collection vessel.

In some embodiments, the bioreactor vessel can be a perfusion bioreactor, wave bioreactor, cylindrical bioreactor, bag bioreactor, moving bed bioreactor, packed bed bioreactor, fibrous bioreactor, membrane bioreactor, batch bioreactor, continuous bioreactor or combinations of the foregoing. In some embodiments, the bioreactor vessel can be made from or comprise a suitable material, for example, stainless steel, glass, aluminum, or plastic. In some embodiments, the bioreactor vessel can allow for analysis of products.

Many past proposals for bioreactors use fluidized beds. While such beds may work well for promoting cell growth and provide certain advantages, the resulting volume of space in the bioreactor vessel required to create such a bed is large. Readily scaling a bioreactor with an unstructured or fluidized bed while achieving the desired cell growth is also challenging, and there is a current demand for bioreactors that may be utilized in a variety of operating conditions in the field (including, for example, within a sterile hood, cabinet or isolator where clearance may be limited).

In some embodiments, the bioreactor vessel described herein comprises a fixed bed. In some embodiments, the fixed bed is a structured fixed bed (which means that it is formed of an easily replicated, generally homogeneous, substantially fixed structure, and thus is not randomly oriented or unstructured, and, as can be appreciated, could take a variety of sizes or shapes while meeting this qualification).

In some embodiments, the structured fixed bed comprises a stack of substrate disks. The substrate layers of the disks are stacked with the first or second side of a substrate layer facing a first or second side of an adjacent substrate layer. In some embodiments, the structured fixed bed extends spirally around a tubular part. In some embodiments, the structured fixed bed described herein can provide for a large cell growth surface within a small volume while still allowing circulation of medium and cells. In some embodiments, the structured fixed bed can be a mesh or comprises a mesh structure. In some embodiments, mesh structure or mesh can be a structure comprising a network or web-like pattern of filament, wire or thread. In some embodiments, the network can define pores, openings or perforations formed of a three-dimensional weave. In some embodiments, the structured fixed bed described herein can comprise a tortuous path for cells and cell culture media. In some embodiments, the tortuous path or channel formed creates turbulence which facilitates cell and cell medium incursion into and/or through the structured fixed bed. In some embodiments the mesh structure is a cell immobilization structure. In some embodiments the mesh structure is or forms a spacer layer or section for flow of cells and medium. In some embodiments the mesh structure is both a cell immobilization and a spacer layer section.

In some embodiments, a spacer layer facilitates the tortuous path. In some embodiments, the structured fixed bed can comprise one or more cell immobilization layers having a surface which allows cells to adhere and grow upon and forming a cell immobilization section. In some embodiments, adjacent to the cell immobilization layers are one or more spacer layers. In some embodiments, the spacer layer can include a structure which forms a spacer section. In some embodiments, the spacer section allows passage of cells and medium through an open but tortuous path. In some embodiments, the structure or nature of the spacer layers can be chosen such that the spacer layers create a tortuous, open path for cells and culture media to travel in parallel to the surface of said spacer and cell immobilization layers. In some embodiments, the tortuous path or channel formed by the spacer section creates turbulence which facilitates cell and cell medium incursion into the immobilization layers.

In some embodiments, the spacer layer can be a mesh or comprises a mesh structure. In some embodiments, mesh structure or mesh can be a structure comprising a network or web-like pattern of filament, wire or thread. In some embodiments, the network can define pores, openings or perforations formed of a three-dimensional weave. In some embodiments, the spacer layers and/or the cell immobilization layers of a spacer section and a immobilization section can be made of a biocompatible polymer, for example polyester (for instance Polyethylene terephthalate (PET)), polyethylene, polypropylene, polyamide, plasma treated polyethylene, plasma treated polyester, plasma treated polypropylene or plasma treated polyamide. In some embodiments, the spacer layer or the cell immobilization layer can comprise silica, polystyrene, agarose, styrene divinylbenzene, polyacrylonitrile or latex. In some embodiments, the layers can be hydrophilic or hydrophobic. In some embodiments, the cell immobilization layer can be hydrophilic. In some embodiments, a cell immobilization layer can be woven or nonwoven. In a preferred embodiment, the spacer layer is made from polypropylene, whereas the non-woven cell immobilization layer is made from hydrophilized PET. In some embodiments, a cell immobilization section and a spacer section can be alternately positioned. In some embodiment, alternately positioned sections can alternate in a vertical position or in a horizontal position. In some embodiments, cell immobilization sections may be layered or alternately positioned in a vertical position or in a horizontal position. In some embodiments one or more layers may be connected. In some embodiments, one or more layers of cell immobilization layers can be superimposed on one or more spacer layers (or vice versa). In some embodiments, a structured bed disclosed herein can be tightly or loosely rolled to a structure such as a spiral structure, a monolith structure or varying shape or could be formed of layers one on top of one another with fluid flowing in parallel or perpendicular to the surfaces of the layers.

In some embodiments, the fixed bed growth surfaces may range from 0.1 m² to 2 m², 7-30 m², 150-600 m², 2,400 m², and may vary among different sizes (height or diameter) of bioreactor vessels. As noted, a plurality of fixed beds may be provided in a stacked configuration, such as one, two, three, four, or more fixed beds. In an embodiment, said fixed bed growth surface has a surface of between 10 to 800 m², more preferably 200 m² to 600 m².

In some embodiments, one or more bioreactor vessel parts are flexible. In some embodiments, one or more bioreactor vessel parts are rigid. In some embodiments, one or more of the bioreactor vessel parts comprise polycarbonate. In some embodiments the one or more bioreactor vessel parts comprise rigid polycarbonate. In some embodiments, the bioreactor vessel comprises polycarbonate. In some embodiments, one or more bioreactor vessel parts are injection molded.

In an embodiment, a bioreactor vessel is provided which may be in modular form, that utilizes one or more structured fixed beds to promote ease of manufacturing and use, while still achieving excellent cell culturing outcomes from the resulting homogeneity and repeatability afforded, even when scaled up or down.

In some embodiments, the modular bioreactor vessel comprises a base portion having a first chamber, an intermediate portion forming at least part of a second, outer chamber for receiving the fixed bed and at least part of a third inner chamber for returning fluid flow from the second outer chamber to the first chamber, and a cover portion for positioning over the intermediate portion.

The fixed bed may comprise a structured fixed bed, and the intermediate portion may comprise a tubular part, the structured fixed bed extending spirally around the tubular part, or the intermediate portion may comprise an inner wall of the fixed bed. In any embodiment, the intermediate portion may comprise a plurality of intermediate parts, each associated with a structured fixed bed.

In some embodiments, at least one of the plurality of intermediate parts is perforated for allowing fluid to flow from a first structured fixed bed below the at least one intermediate part to a second structured fixed bed above the at least one intermediate part. In some embodiments, each of the plurality of intermediate parts is tubular, and each structured fixed bed comprises a spiral bed wound around the tubular intermediate part. A perforated support may be provided for the structured fixed bed.

In some embodiments, the intermediate portion may further comprise a tubular casing for forming a periphery of the modular bioreactor vessel. The tubular casing forms a space for heating, cooling, or insulating the bioreactor vessel. The intermediate portion may comprise a plurality of intermediate parts, each adapted for connecting with each other.

In some embodiments, the intermediate portion includes a tube for engaging at least one intermediate part and forming an inner wall of the outer second chamber for receiving the fixed bed. The tube may engage wherein the tube engages a first intermediate part below the tube and a second intermediate part above the tube. The second intermediate part may include openings for creating a fluid film along the third inner chamber. Supports, such as vertical rods, may be provided for supporting the second intermediate part from the first intermediate part.

In some embodiments, the cover portion comprises a cap including a plurality of ports. In some embodiments, the cover portion comprises a removable cap. The removable cap may have an outer diameter that is less than an outer diameter of the intermediate portion. The removable cap may have an outer diameter that is more than an outer diameter of the intermediate portion. At least one of the ports may include a threaded metal insert. The cover portion may have an outer diameter that is equal to or greater than an outer diameter of the intermediate portion.

The intermediate portion may comprise an intermediate part adapted for positioning at least partially within the base portion. The intermediate part may further include a flow disruptor for disrupting fluid flow.

The base portion may include a further chamber radially outward of the first chamber in fluid communication with the second outer chamber including the fixed bed. This further chamber may be formed in part by an upstanding wall having a plurality of openings for transmitting fluid from the first chamber to the further chamber.

In some embodiments, an agitator is associated with the base portion. The intermediate portion may be adapted for suspending the agitator in the first chamber in a manner that allows side-to-side movement for alignment with an external drive.

In some embodiments, a container is provided for containing the agitator. In some embodiments, the container includes a central inlet and a plurality of radially oriented outlets. A flow divider may be associated with the central inlet. In any embodiment, or as an independent component separate from any bioreactor vessel, the agitator may comprise a plurality of curved blades.

In some embodiments, a plurality of flow disruptors are provided for dividing the fluid flow entering the third inner chamber into a plurality of streams. The plurality of flow disruptors may be associated with a ring. In some embodiments, one or more conduits for permitting gas to enter into a space behind one of the streams are provided. The one or more conduits may be connected to a structure including the plurality of flow disruptors. For example, a first conduit may be connected to the structure, or both first and second conduits may be connected to the structure. Alternatively, the first and second conduits may not be connected to the structure.

In another or further embodiment, the system is provided with a bioreactor vessel, the bioreactor vessel including a fixed cell culture bed and an agitator for pumping liquid through the cell culture bed, wherein the agitator is placed in a container. In an embodiment, the agitator is connected to a conduit. In an embodiment, the conduit comprises an injector for delivering gas bubbles into the container. In an embodiment, the agitator converts the bubbles originating from the injector into second bubbles having a second, smaller size than the first size for delivery to the cell culture bed with the liquid. Given their smaller size, the second bubbles are better able to pass into and through channels formed by the spacer layers and the adjacent cell immobilization layers (or other available paths) of the fixed bed. This serves to further enhance the oxygenation of the cells being grown in the bed, without a corresponding need to increase the speed of the impeller and the resulting liquid flow rate. Moreover, the release of the gas into or near the agitator container and the resulting flow avoids the creation of deleterious air pockets in the bioreactor vessel, which are notoriously difficult to remove without halting the bioreactor vessel operation.

In an embodiment, the bioreactor may include a support for supporting the fixed bed. In one form, this support may comprise a container for containing the agitator, such as impeller, in an interior compartment of the housing. The container may be adapted to receive fluid from a central opening and eject the fluid radially outwardly via one or more openings (e.g., four spaced 90 degrees apart), such as a result of the movement (rotation) of the agitator, such as impeller. The container may further include one or more outward projections, which serve as positioners for centering or uniformly spacing the container from an inner wall of the housing, but without being attached to it. For example, the container along an upper portion may include one or more radially extending arms. These arms may be adapted for aligning or centering the container within the housing of the bioreactor when rested on a surface thereof, such as the floor. While the arms may be on the container, the arms may instead attach to the inner wall of the housing and extend toward the container, but not attach to it, to facilitate easy removal.

In an embodiment, the bioreactor includes a housing having a wall defining an interior compartment, a plurality of fixed beds for culturing cells, and a plurality of annular fixed bed supports. Each of the plurality of fixed bed supports is adapted to support a respective at least one of the plurality of fixed beds. Each of the plurality of fixed bed supports comprises an annular section and a support frame extending radially out from the annular section. The support frame has an outer diameter corresponding in size to an inner diameter of the wall of the housing, said support frame being adapted to support at least one of the plurality of fixed beds from underneath and to allow fluid to flow through the support frame. The plurality of fixed bed supports are adapted to interlock with one another to form a peripheral chamber between the plurality of annular fixed bed supports and the wall of the housing, as well as a central chamber within the annular sections. The bioreactor further includes a lid for connecting to the housing and for sealing the plurality of fixed beds and the plurality of fixed bed supports in the interior compartment, a plurality of probes extending into the interior compartment adjacent to or into at least one of the fixed beds, and an upper frame overlying the plurality of fixed bed supports and forming a plurality of pockets for allowing fluid to accumulate therein upon exiting an upper end of the plurality of fixed beds. At least one of the plurality of probes is adapted for sensing a characteristic of the fluid in a respective one of the plurality of pockets. The bioreactor further includes an impeller for circulating fluid within the bioreactor and a container for containing the impeller. The container comprises a plurality of openings adapted to allow fluid to flow from within the container to the peripheral chamber, and a plurality of positioners in the form of radially extending arms extending therefrom and adapted to position the container within the housing and space the container from the wall thereof. The upper frame is adapted to interlock with at least one of the plurality of annular fixed bed supports and to interlock with the lid for preventing relative rotation therebetween.

In an embodiment, the bioreactor includes a housing having a wall defining an interior compartment, a removable fixed bed for culturing cells, and a removable fixed bed support adapted to support the fixed bed. The fixed bed support is annular in shape and includes a plurality of arms extending radially outward, the radially extending arms defining an outer diameter corresponding in size to an inner diameter of the wall of the housing for positioning and centering the fixed bed support in the housing. The plurality of arms are adapted to support the fixed bed from below. The fixed bed support forms a peripheral chamber between an outer wall of the fixed bed support and the housing, as well as a central chamber within the fixed bed support. The fixed bed is adapted to be positioned within the peripheral chamber. The housing includes one or more receivers in the wall of the housing for receiving at least one of the plurality of arms, the one or more receivers adapted to support the fixed bed support within the interior compartment and to prevent relative rotation of the fixed bed support within the housing. The bioreactor further includes a lid for connecting to the housing and for sealing the fixed bed and the fixed bed support in the interior compartment, and at least one probe extending into the interior compartment at a location within the peripheral chamber and above the fixed bed. The bioreactor further includes an impeller adapted to rotate on an impeller support, the impeller for circulating fluid within the bioreactor. The impeller is located in a chamber formed between a lower portion of the fixed bed support and a floor of the housing. The impeller is adapted to circulate fluid from the central chamber of the fixed bed support and outward to the peripheral chamber and up through the fixed bed therein. The bioreactor further includes a drain tube connected to the impeller support for draining the liquid from the bioreactor.

In an embodiment, said bioreactor vessel is a single-use bioreactor vessel, said collection vessel is a single-use collection vessel and/or said pressure sensors are single-use pressure sensors. The general purpose of the collection vessel is to have a gamma irradiated “plug and play/ready to use” solution. In an embodiment the collection vessel has been designed in polypropylene with the possibility to hang a TFF on the side using holders. The TFF selected is gamma irradiated and with a complete gamma stable manifold (collection vessel+pump+TFF). In a preferred embodiment, the fluid path within the system is a fully closed system made of disposable consumables (e.g. bioreactor vessel, filters, TFF membranes, bottles, sampling devices, single used sensors) interconnected by disposable tubing manifolds. In an embodiment, the fluid path includes sampling systems. In an embodiment, a foam trap is connected with the collection vessel. In an embodiment, the bioreactor vessel is developed for single-use and comprises disposable pre-fitted manifolds for a top and bottom liquid bioreactor vessel drain, a liquid sample line, a bubble or foam trap and a base addition.

In order to be able to fully operate and execute the task of producing or purifying the desired biomolecules, said bioreactor vessel may be comprised in a bioreactor cabinet, said bioreactor cabinet being adapted to dock into said system.

Said bioreactor cabinet is preferably a wheeled (or otherwise mobile) bioreactor cabinet suited to receive a bioreactor vessel, said bioreactor cabinet is provided thereto with a bioreactor docking station. Said bioreactor cabinet, preferably a side wall of said bioreactor cabinet, is provided with a connector allowing the transmission of power, signals and/or data when paired with the biomolecule production system, such as a bioreactor chamber of said system.

In one embodiment, a connection between the bioreactor cabinet and the system will allow docking of the bioreactor cabinet to the system and ensures that both entities are firmly connected to each other, prohibiting the release of the bioreactor cabinet from the system during the production of biomolecules. In a preferred embodiment the connection is magnetic. Said magnet may be an electro magnet, wherein magnetic field is produced by an electric current. The main advantage of an electromagnet over a permanent magnet is that the magnetic field can be quickly changed by controlling the amount of electric current. In the current application, the use of a magnet, more specifically an electro magnet enhances the safety of the system, as it will prevent unauthorized docking or removal of the bioreactor cabinet to or from the production system. Said system may be comprised of a corresponding magnetic part to allow interaction with the magnet of said bioreactor cabinet. In an embodiment, said magnetic connection is controlled by the software.

In order to allow docking and functioning, the bioreactor cabinet is also provided with a connector allowing the transmission of power, signals and/or data when paired with a biomolecule production system and a connection, preferably magnetic, for allowing the connection to said biomolecule production system. In an embodiment, said connector may be comprised of a connecting portion and receiving portion, wherein said connecting portion may be present on said bioreactor cabinet and said receiving portion may be present at a recess in said bioreactor chamber; or vice versa. In an embodiment, the powered part of the magnetic connection is provided on the system and the stainless steel part of the magnetic connection is provided on the back of the bioreactor cabinet. In an embodiment the 2 parts become blocked once power is added. In an embodiment, the 2 parts are blocked with a force of 1000N.

In an embodiment, a male connector of the bioreactor cabinet is connected to a female connector of the production system. In a preferred embodiment, to ensure correct connection between the male and female connector, the female connector contains centering pins.

In a preferred embodiment, the connector can be a modular connector system allowing combinations of power and signal contacts, Ethernet, optical fiber, coaxial contacts, hydraulic, pneumatic and thermocouplings in a compact frame or housing. This modular connector system can be configured according to the specific requirements of the connection. In a preferred embodiment the connectors are waterproof.

The bioreactor cabinet will be connected to the bioreactor chamber with an industrial connector providing a reliable and pluggable transmission of the power, signal and data.

Traditionally, the production of biomolecules, such as biopharmaceuticals requires steel-based bioreactors. These need to be thoroughly cleaned and sterilized for the production of contamination-free bioproducts, thus increasing costs for the manufacturer. In recent years, single-use systems comprising for instance single-use vessels have become a more and more established standard in the biopharma industry, which comes as no surprise. After all, they offer a range of advantages like flexibility, lower costs and reduced energy consumption.

In a further aspect, the current disclosure relates to a biomolecule production system for producing biomolecules, wherein said system comprises one or more single-use vessels, wherein at least one of the one or more single-use vessels is equipped with at least a first and a second pressure sensor for determining the volume of liquid in said vessel, said first pressure sensor measuring the hydrostatic pressure in the vessel and said second pressure sensor measuring the gas headspace pressure (such as the air pressure) in said vessel. Said one or more single-use vessels could be any type of vessel suitable for use in the biomolecule production system, such as a bioreactor vessel or a collection vessel.

In an embodiment, in addition to the vessels, also the pressure sensors can be single-use.

Single-use facilities are easier to maintain. Costs for complex production stages like cleaning (CIP) and sterilization (SIP) become void when using single-use technologies, thus saving costs and resources.

Those direct savings in terms of material and labor costs are one of the main advantages of single-use systems. Direct labor costs for installation as well as costs for water and chemicals can be minimized. Furthermore, the facilities do not require cleaning and sterilization, which in turn leads to a prolonged operating life and further reduction of total cost of ownership. In addition, single-use systems help reduce initial investment as well as R&D costs, which is a huge advantage given the ever increasing demand for biopharmaceuticals. The initial investment costs are approximately 40% lower than the price for a comparable stainless-steel facility. As single-use systems are disposable, they do not require any elaborate cleaning and disinfection, but can rather be disposed of immediately after their utilization.

Furthermore, the transition from traditional devices to single-use systems leads to an obvious decline in energy and water consumption. Biopharmaceutical production facilities using single-use technologies could reduce their overall water and energy consumption by 46% when compared to stainless-steel reactors. Additionally, single-use facilities have a 35% lower CO₂ footprint than stainless-steel reactors.

In general, single-use systems are designed and dimensioned for disposable liquid pathways, which allows for quick and easy installation. This saves time and costs in terms of preparation, implementation, validation and documentation.

The prevention of cross-contamination is amongst the biggest challenges that the biopharma industry faces. The risk of contamination is particularly high if different antibodies and/or proteins are manufactured in the same facility. Contamination leads to a loss of drug substance and requires additional cleaning steps. In the worst case, cross-contamination can lead to a potentially lethal treatment of patients. With the disposal of the liquid pathway after each batch, single-use technology helps to overcome this challenge-like this, cross-contamination becomes practically impossible.

In a further aspect, the invention relates to a method for measuring the volume of liquid in a vessel, wherein said vessel is equipped with at least a first and second pressure sensor, said first pressure sensor measuring the hydrostatic pressure in the vessel and said second pressure sensor measuring the gas headspace pressure in said vessel and wherein the volume of liquid in said vessel is calculated based on said pressure measurements.

In a further aspect, the current disclosure relates to a method for producing a biomolecule, such as a protein, a virus or viral particle, or gene therapy product by means of a system according to any of the embodiments as described above.

In a further aspect, the invention relates to a method for producing a biomolecule, such as a protein, a virus or viral particle, or gene therapy product, comprising the steps of providing a biomolecule production system comprising one or more vessels, such as a bioreactor vessel, a transfer vessel, a reagent vessel, a waste vessel and/or a collection vessel, and optionally a concentrator, wherein one or more of said vessels are equipped with at least a first and second pressure sensor, said first pressure sensor measuring the hydrostatic pressure in the vessel and said second pressure sensor measuring the gas headspace pressure in said vessel and wherein the volume and/or weight of liquid in said vessel is calculated based on said pressure measurements. In an embodiment, the biomolecule production system comprises a bioreactor vessel equipped with at least a first and second pressure sensor. In an embodiment, the biomolecule production system comprises a collection vessel, wherein the collection vessel is equipped with at least a first and second pressure sensor. In an embodiment, the biomolecule production system comprises a transfer vessel, wherein the transfer vessel is equipped with at least a first and second pressure sensor. In an embodiment, the biomolecule production system comprises a reagent vessel, wherein the reagent vessel is equipped with at least a first and second pressure sensor. In an embodiment, the biomolecule production system comprises a waste vessel, wherein the waste vessel is equipped with at least a first and second pressure sensor. In an embodiment, the biomolecule production system comprises a bioreactor vessel, a transfer vessel, a reagent vessel, a waste vessel and/or a collection vessel, wherein one or more of said vessels are equipped with at least a first and second pressure sensor. In an embodiment, the biomolecule production system further comprises a concentrator.

In a preferred embodiment, the invention relates to a method for producing a biomolecule, such as a protein, a virus or viral particle, or gene therapy product, comprising the steps of providing a biomolecule production system comprising a bioreactor vessel, a collection vessel, a concentrator and a waste vessel, collecting the harvest from said bioreactor vessel in a collection vessel and further concentrating said harvest by means of a concentrator, wherein one or more of said vessels are equipped with at least a first and second pressure sensor, said first pressure sensor measuring the hydrostatic pressure in the vessel and said second pressure sensor measuring the gas headspace pressure in said vessel and wherein the volume and/or weight of liquid in said vessel is calculated based on said pressure measurements.

In an embodiment, said method further comprises controlling the liquid flow between said one or more vessels and optionally said concentrator based on the measurement of the volume of liquid (or the weight of the liquid) in said vessels, wherein said liquid flow is controlled by means of pumps and valves.

In an embodiment, said method further comprises controlling the liquid flow between said bioreactor vessel, collection vessel, concentrator and waste vessel based on the measurement of the volume of liquid (or the weight of the liquid) in said vessels, wherein said liquid flow is controlled by means of pumps and valves.

In a further embodiment, the functioning of the pumps and valves is controlled by a process controller, such as a Programmable Logic Controller (PLC), which is connected to and receives data from said pressure sensors.

In a further embodiment, said process controller detects that a certain threshold liquid or weight level is reached in a certain vessel and automatically controls the liquid flow by means of pumps and valves to said vessel.

In an embodiment, said process controller automatically prevents overfilling of said vessel (see for instance FIG. 25A).

In an embodiment, said process controller automatically maintains a constant volume or a constant concentration of biomolecules in said vessel, for instance during one or more diafiltration or clarification steps of the harvest from the bioreactor vessel. In an embodiment, said process controller automatically maintains a constant volume or a constant concentration of biomolecules in said vessel during a perfusion step.

In an embodiment, said process controller automatically starts and/or halts a concentration step of the bioreactor vessel harvest by controlling the pumps and valves controlling liquid flow between the collection vessel, concentrator and waste vessel, thereby obtaining a predetermined biomolecule concentration (see for instance FIGS. 23 and 24A-B, showing a schematic representation of such a cycle).

In an embodiment, said process controller automatically starts and/or halts draining of the vessel (see for instance FIG. 25B).

In an embodiment, the concentration of biomolecules in said one or more vessel is determined based on the measured volume of liquid in said one or more vessels.

Said method may comprise the steps of providing a bioreactor vessel, wherein a harvest from said bioreactor vessel is filtered or purified to produce a biomolecule harvest, said biomolecule harvest is further concentrated by means of a concentrator and collected in a collection vessel, wherein the volume of liquid in one or more of said vessels is determined by means of a first pressure sensor measuring the hydrostatic pressure in the vessel and a second pressure sensor measuring the gas headspace pressure (for instance the air pressure) in said vessel. In an embodiment, said bioreactor vessel, concentrator and collection vessel are present in a bioreactor chamber. In an embodiment, said harvest from said bioreactor vessel is filtered or purified in a process chamber. In an embodiment, said biomolecule harvest is in-line clarified in one or more filters present in a downstream chamber flanking said bioreactor chamber.

A possible process flow in an embodiment of the system involves the production of a biomolecule, such as a viral particle, e.g. for producing a vaccine or a viral gene therapy product. To this purpose cells are cultured in the bioreactor vessel inside a bioreactor cabinet which is embedded in the bioreactor chamber. Media and buffer are supplied to the bioreactor vessel by means of externally supplied bags, that are connected to the bioreactor chamber. Waste that is produced during the production cycle is guided towards a waste vessel. Subsequently the bioreactor harvest is lysed and transported to a process chamber, where it is filtered using purification or filtration devices. After this step, the product is either harvested or transported to the bioreactor chamber, where it is concentrated by means of the collection vessel and TFF, wherein the volume of liquid in one or more of said vessels is determined by means of a first pressure sensor measuring the hydrostatic pressure in the vessel and a second pressure sensor measuring the gas headspace pressure (for instance the air pressure) in said vessel. Afterwards, the concentrate is transported towards the purification or filtration devices in the downstream chamber. Additional chambers can be connected to said system in case further upstream or downstream processing is needed.

In a further aspect, the current disclosure relates to a method for producing a biomolecule, such as a protein, a virus or viral particle, or gene therapy product, comprising the steps of providing a bioreactor vessel provided in a bioreactor chamber of a biomolecule production system, and wherein a harvest from said bioreactor vessel is clarified in a processing chamber flanking said bioreactor chamber to produce a biomolecule harvest, said biomolecule harvest is collected in a collection vessel and further concentrated by means of a concentrator located in said bioreactor chamber, wherein one or more of said vessels are equipped with at least a first and second pressure sensor and the volume of liquid in said vessel is measured by means of said pressure sensors.

Processes in the bioreactor vessel produce biomolecules from cultured cells. The resultant product is optionally purified in the process chamber which is fluidly connected and adjacent to the bioreactor chamber. In an embodiment, the cultured cells are lysed before further processing. In an embodiment, DNA is removed from the cultured cells before further processing. This process chamber comprises one or more purification, clarification or filtration devices allowing the purification or filtration of a biomolecule of a cell harvest.

Afterwards, the product is either harvested or transported to the bioreactor chamber, where it is concentrated by means of the collection vessel and TFF, wherein the volume of liquid in one or more of said vessels is determined by means of a first pressure sensor measuring the hydrostatic pressure in the vessel and a second pressure sensor measuring the gas headspace pressure (for instance the air pressure) in said vessel. Afterwards, the concentrate is transported towards the purification or filtration devices in the downstream chamber. In an embodiment, said downstream chamber is in fluid connection with said bioreactor chamber and/or the process chamber. In a preferred embodiment, said downstream chamber is in fluid connection with said bioreactor chamber. In a further embodiment the purification or filtration devices in the downstream chamber are first flushed to a waste tank before seeing product. After the flushing, the product in the collection vessel is sent through the devices in the downstream chamber until the collection vessel is emptied. In a further embodiment, the devices are then rinsed with a chasing buffer for a specified time. The chasing step is combined with the rinsing cycle of the collection vessel and the TFF. Buffers are located outside of the system and are introduced in the system by the left side as for the media. In some embodiments, a specific pump is scheduled to add buffers. In an embodiment, an assembly (named bioharvest feed assembly) is present for transfer of the harvest from the process chamber to the collection vessel and a waste vessel.

In another embodiment, an assembly (named diafiltration buffer assembly) is present, starting from a diafiltration buffer feed in the process chamber to the collection vessel in the bioreactor chamber and the secondary clarification system in the downstream chamber or from the collection vessel in the bioreactor chamber to the secondary clarification system in the downstream chamber. In an embodiment, this diafiltration buffer assembly is connected to a buffer feed assembly, wherein this buffer feed assembly provides buffer for the clarification filters for filter wetting/priming. In an embodiment, the buffer feed assembly comprises four inlet connections and a single-use pump head.

In another embodiment, an assembly (named permeate/waste assembly) is present connecting the TFF permeate and the waste vessel in the downstream chamber. In another embodiment, an assembly (named bulk product outlet assembly) connects the secondary clarification assembly or the diafiltration buffer assembly with the waste vessel and the transfer bag.

In an embodiment, fluidly connected includes one or more intervening manifolds, vessels, devices etc.

In a particular embodiment, the current disclosure provides for a system and method for the production of a (therapeutic) gene therapy product, more preferably a human gene therapy product, even more preferably a viral gene therapy product that uses a viral vector to introduce genetic material in a subject. In an embodiment, said viral vector may be a retrovirus, adenovirus, herpes simplex, vaccinia, lentivirus or an adeno-associated virus.

During the infection phase, the virus is added to the bioreactor vessel. In an embodiment, the virus is added to the bioreactor vessel by means of a virus infection kit. In an embodiment, the virus infection kit comprises a two-part bottle assembly for the virus infection process and two spare connections. In an embodiment, the virus is added to the bioreactor vessel by means of a pump, such as a Watson-Marlow peristaltic pump.

In an embodiment, an endonuclease is added to the bioreactor vessel for nucleic acid removal. In an embodiment, an endonuclease is added to the bioreactor vessel via an inlet for small additions. Endonucleases are the ideal tool for nucleic acid removal in virus vector and vaccine manufacturing. In a further embodiment, the endonuclease is added by means of an endonuclease assembly comprising a 5 L single-use bottle assembly and extra connections. In an embodiment, the endonuclease is added to the bioreactor vessel by means of a pump, such as a Watson-Marlow peristaltic pump. In an embodiment, the endonuclease is benzonase endonuclease, which degrades both DNA and RNA to small 3-5 base pairs (<6 kDa) fragments with no base preference. The use of benzonase endonuclease additionally increases the yield in virus purification, protects the downstream chromatography and filter devices from fouling and reduces feed stream viscosity.

In an embodiment, transfection reagent is added to the bioreactor vessel by means of a transfection assembly comprising a single-use bag assembly and two extra connections.

In another embodiment, said biomolecule that is produced is a vaccine, such as a vaccine against influenza, SARS, MERS, COVID-19, Measles, Rabies, Zika, Polio, Mumps or Rubella.

In a further aspect, the current disclosure relates to use of aforementioned system, for the production of biomolecules, such as proteins, viruses and/or viral vaccines.

In an embodiment, aforementioned system is used for the production of a (therapeutic) gene therapy product, more preferably a human gene therapy product, even more preferably a viral gene therapy product that uses a viral vector to introduce genetic material in a subject. In an embodiment, said viral vector may be a retrovirus, adenovirus, herpes simplex, vaccinia, lentivirus or an adeno-associated virus.

In a last aspect, the current disclosure relates to a method for determining the total liquid volume in a vessel of a biomolecule production system (such as a bioreactor vessel a transfer vessel, a reagent vessel, a waste vessel and/or a collection vessel) by means of a process controller, said vessel comprising at least a first and a second pressure sensor coupled to said process controller, wherein said total liquid volume consist of a first volume of liquid below the first pressure sensor and a second volume of liquid above the first pressure sensor, said total liquid volume is determined by: calculating the first volume of liquid and adding this to the second volume of liquid, said second volume of liquid being determined by measuring the hydrostatic pressure by means of the first pressure sensor, measuring the gas headspace pressure (for instance the air pressure) by means of the second pressure sensor, thereby determining the differential pressure and calculating the volume of fluid above the first pressure sensor, wherein the differential pressure is comprised in a range between 0 to 200 mbar.

Said first volume of liquid is simply calculated by determining the volume below the pressure sensor taking the geometry of the vessel into account.

As discussed above, said second volume of liquid can be determined by means of Pascal's principle by using the formula: P_{diff}=ρ× g× h, wherein P_{diff} is the differential pressure as determined by the first and second pressure sensor (hydrostatic pressure measured by the first pressure sensor minus gas headspace pressure (for instance the air pressure) measured by the second pressure sensor), h=height above the first pressure sensor (in m), g=gravity constant (=9.81 m/s²), ρ=fluid density (in kg/m³). After calculation of the height above the first pressure sensor, one can, based on the internal surface of the vessel and the fluid density, calculate the volume above the first pressure sensor. The total volume of liquid in the vessel is calculated by adding the first and second volume of liquid together.

Likewise, as described above, also the weight of the liquid volume can be calculated based on the pressure measurements. In an embodiment, the total weight of the liquid volume in the vessel can be calculated based on the addition of two volumes and can be calculated based on following Equation 3:

### Equation 3

m=ΔP×S/g+b, wherein “ΔP×S/g” is the volume above the first pressure sensor linked to differential pressure and wherein “b” is the “residual” volume below the first pressure sensor that is known by the geometry of the vessel and the location of the first pressure sensor.

### DESCRIPTION OF FIGURES

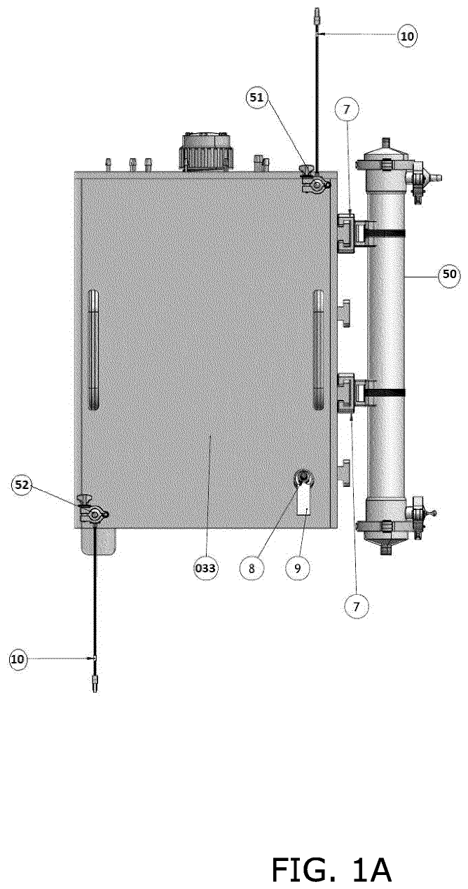

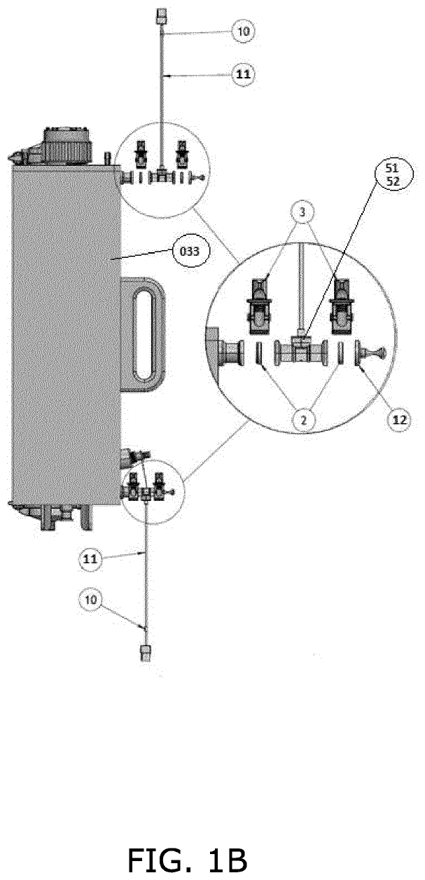

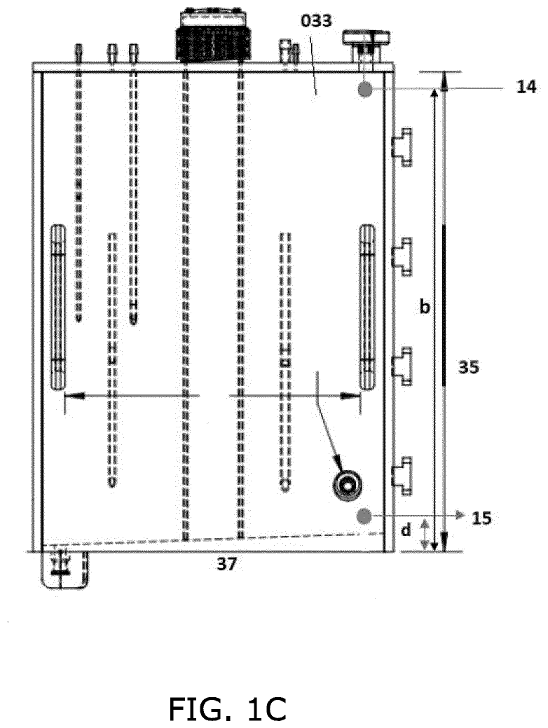

FIGS. 1A-1C illustrate an embodiment of the collection vessel 033 of the system according to the current disclosure. The collection vessel 033 has the shape of a rectangular prism. The biomolecule production system comprises a bioreactor vessel (not shown), a collection vessel 033 and a concentrator, such as a TFF 50, to obtain a heavily concentrated biomolecule (not shown). The retentate of the TFF 50 will be brought back to the collection vessel 033, whereas liquid waste will be discarded (preferably to a waste bottle, not shown). Due to the re-circulation of retentate back and forth from the collection vessel 033 to the concentrator 50, a heavily concentrated biomolecule product will be obtained. Two TFF holders 7 physically attach the TFF 50 to the collection vessel 033. The collection vessel 033 is equipped with a pH probe 8 to measure the pH inside the collection vessel 033. A label 9 is attached to the pH probe 8.

Finally, the recirculated output of the TFF 50 is harvested in the collection vessel 033 thereby obtaining a concentrated cell culture harvest. The presence of a collection vessel 033 offers the advantage that the bioreactor vessel (not shown) can be rinsed to harvest remaining liquid, while the volume of this rinsing liquid can still be reduced by the TFF 50 prior to further downstream processing.

Determination of the liquid level or the liquid weight in the collection vessel 033 is important. As such, the collection vessel 033 is equipped with a first 52 and a second 51 pressure sensor for determining the volume (or weight) of liquid in said collection vessel 033, said first pressure sensor 52 measuring the hydrostatic pressure in the collection vessel 033 and said second pressure sensor 51 measuring the gas headspace pressure in said collection vessel 033. The sensors are electrically connected by means of electrical wiring 11 and an electrical connector 10 to monitors or control systems, such as a process controller (not shown).

The pressure sensors 51,52 are incorporated in a two flanged design. This design is connected by means of tri-clamp gaskets 2 and clamps 3 to the collection vessel 033 and to an end cap 12 as depicted in FIG. 1B. The position of the sensors is important to allow for an accurate measurement. FIG. 1C depicts the collection vessel 033 indicating the position of the first sensor 15 and the position of the second sensor 14. Both sensors 51,52 are positioned on the front side of the collection vessel 033. The location 15 of the first pressure sensor 52 is at a height d which is smaller than 1/10^{th} of the total length 35 of the collection vessel 033, measured from the bottom wall 37 of said vessel. The location 14 of the second pressure sensor is at a height b which is greater than 9/10^{th} of the total length 35 of the collection vessel 033, measured from the bottom wall 37 of said vessel. Said sensors can also be positioned on opposite sides (left and right) of the collection vessel 033 as depicted in FIG. 1A.

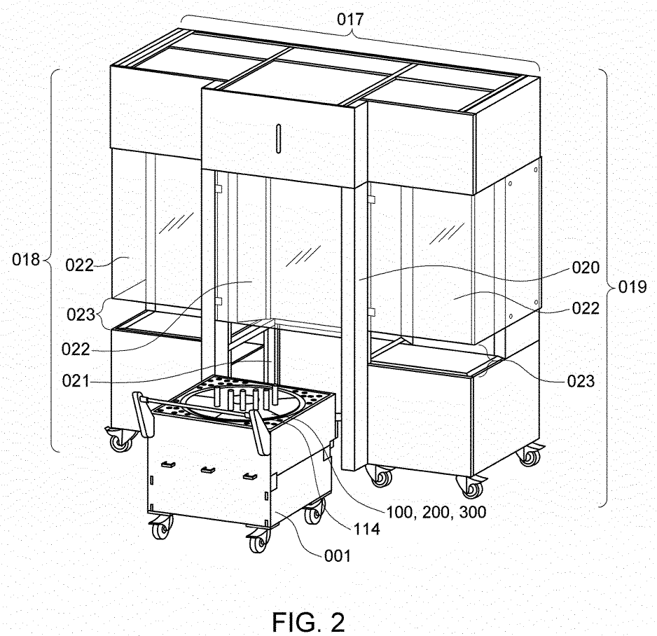

FIG. 2 illustrates a front view of a biomolecule system 017 according to an embodiment of the disclosure showing a process chamber 018, a downstream chamber 019 and a bioreactor chamber 020, suited to receive a bioreactor vessel 100,200,300 in a bioreactor cabinet 001. To that purpose the system 017 is provided with a recess 021 that allows receiving a bioreactor cabinet 001 in said system 017. The bioreactor chamber 020 is mandatory; the process chamber 018 and downstream chamber 019 are optionally and can be coupled separately to the bioreactor chamber 020. The bioreactor vessel 100,200,300 includes an external casing or housing 112 (not shown) forming an interior compartment and a removable cover or top surface 114 for covering the interior compartment. The user handling area of each chamber in the system is shielded with a front window 022, said front window is preferably provided with a gap 023 to allow access during operation.

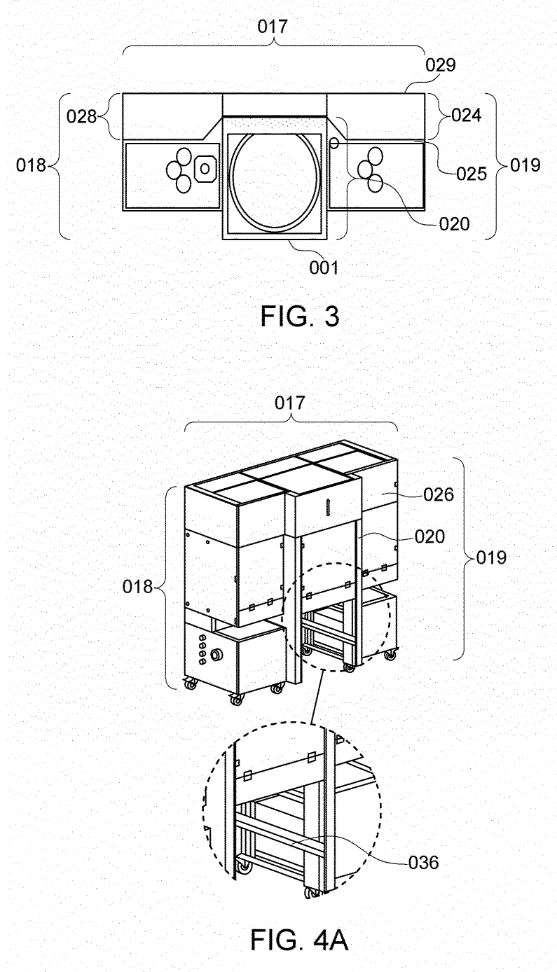

FIG. 3 shows a top view of a system 017 according to an embodiment of the current disclosure comprising a process chamber 018, a downstream chamber 019 and a bioreactor chamber 020. The bioreactor cabinet 001 of the system 017 when fitted in said system protrudes from the processing chamber 018 plane, allowing the operator easier access to the bioreactor cabinet 001. The back of each chamber in the system is provided with an electrical cabinet 024. These electrical cabinets supply the power to instruments and control the process and are composed by a back sheet 025, a technical enclosure 028 and a space for a circuitboard 029. In this embodiment, the electrical cabinets are made of stainless steel and are accessible by opening the back doors of the system. The back sheet 025 is fixed to the front of the electrical cabinet 024 to allow proper instrument fixation and electronic part dissimulation. Key components to be accessible by maintenance during operations are located in the front part of the chamber, while all terminals boxes, wiring and electronics are in the back with no access during the operation.

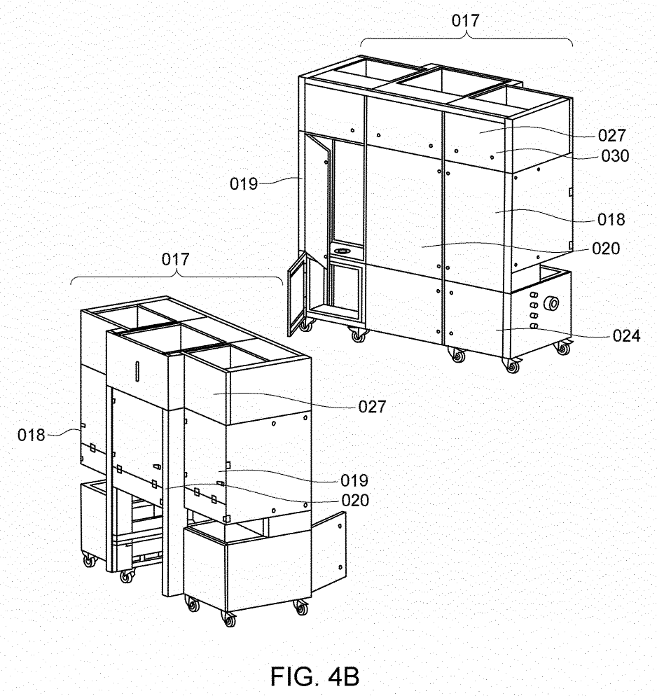

FIG. 4A shows a front view of a system 017 according to an embodiment of the current disclosure. The system comprises a process chamber 018 comprising one or more purification or filtration devices allowing the purification or filtration of a biomolecule of a cell harvest, a downstream chamber 019 and a bioreactor chamber 020. The bioreactor chamber is suited to receive a bioreactor vessel 100,200 in a bioreactor cabinet 001. The bioreactor cabinet 001 is guided into the bioreactor chamber by the use of guides 036 on the bioreactor chamber 020 and wheels 013 on the bioreactor cabinet 001. The general casing 026 is the main structure of the system. In some embodiments, the length of the system 017 can be reduced depending on the number of filters in the process chamber 018 and the downstream chamber 019. Materials used for the general casing 026 of said system are resistant to corrosion. In the embodiment shown in FIG. 4A, the metallic elements are made of stainless steel SS316 with a rugosity of Ra≤1.2 μm.

FIG. 4B shows a back view and a front view of the system 017 shown in FIG. 4A. A HVAC system 027 is placed on top of the chambers 018,019,020 ensuring that air with the appropriate quality level is supplied to the system chambers. The backside and sides of the casing of said system is comprised of electrical 024 and pneumatic cabinets 030, comprising the important electrical and pneumatic components of said system. This allows easy access to these electrical and pneumatic components by the operator, meanwhile ensuring that the operator does not have to enter the spaces wherein the biomolecules are produced.

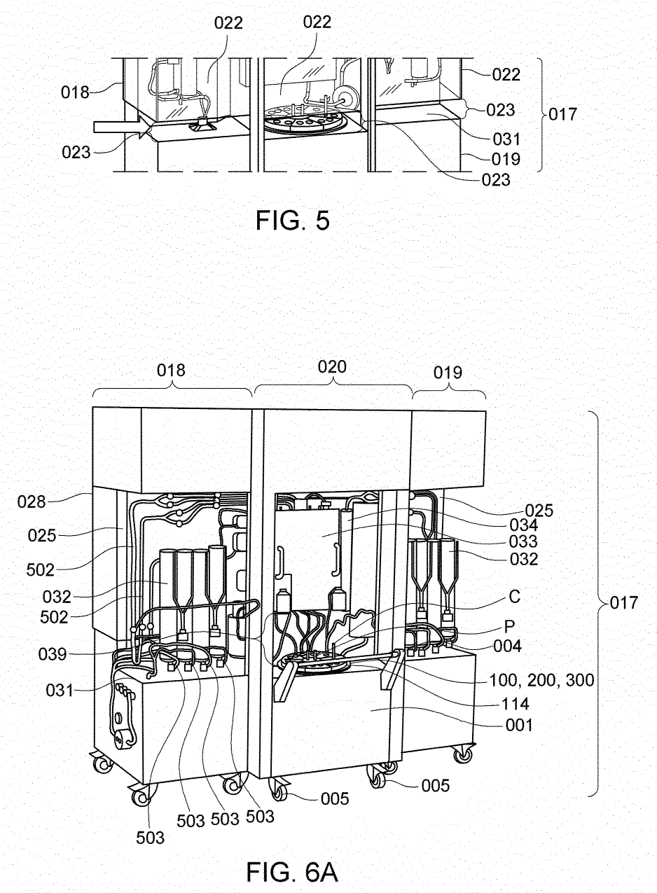

FIG. 5 illustrates a detail of the front view of a system according to an embodiment of the current disclosure including the front windows 022. In a preferred embodiment the general casing of the system has front windows with a gap 023 of 200 mm with the work plan 031. This gap allows the evacuation of the air from the process chamber 018 and gives access during operations. The overall design allows the operators to stay in front of the chamber. In an embodiment the windows can be opened by two different ways (vertically and horizontally).

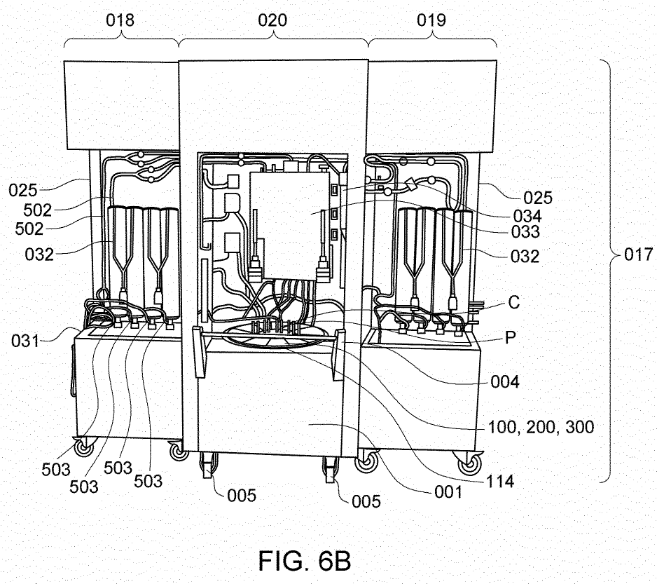

FIG. 6A illustrates a preferred embodiment of a system 017 according to the current disclosure. In this embodiment, a bioreactor chamber 020 is positioned centrally in the production system 017 which is flanked by a process chamber 018 and a downstream chamber 019. This bioreactor chamber 020 allows docking of the bioreactor cabinet 001 comprising a bioreactor vessel 100,200,300. To that purpose, the bioreactor chamber 020 is provided with a recess (not shown) that allows receiving said bioreactor cabinet 001. To facilitate docking of the bioreactor cabinet a handle 004 is present on the bioreactor cabinet 001. The bioreactor cabinet 001 contains wheels 005 to allow easy transportation. The bioreactor vessel 100,200,300 includes an external casing or housing (not shown) forming an interior compartment and a removable cover or top surface 114 for covering the interior compartment, which may include various openings or ports P with removable covers or caps C for allowing for the selective introduction or removal of fluid, gas (including by way of a sparger), probes, sensors, samplers, or the like.

Bioreactor harvest from the bioreactor ports will be transported to the process chamber 018, the appropriate pipings 039 are provided to allow fluid transfer. The process chamber 018 is provided with a one or more purification or filtration devices 032 allowing the purification or filtration of a biomolecule of a cell harvest. The purification or filtration devices 032 are provided with an outlet line having a vertical section 502 parallel to said purification or filtration devices 032, and which allows for safe priming and venting of said purification or filtration devices 032 prior to use.

Priming solution is providing via an inlet line 503 in connection to said purification or filtration devices 032. Such filter may for instance be an in-depth filtration system. The number of filters in the process chamber 018 is flexible, depending on the product that is to be produced. As the filters are located on the side of the system, the design is quite flexible if a huge number of filters must be added. The working space 031 is located at around 90 cm from the ground to allow the operators to perform procedures standing up.

The background metal sheet 025 in the process chamber 018 is designed with all equipment's and devices accessible by the operators, while in the back 028 all technical components are installed like motors, network cables, power supply, etc.

The bioreactor chamber 020 is equipped with a collection vessel 033 and a TFF 034 in order to concentrate the harvest. The collection vessel 033 and TFF 034 are fluidly connected to each other. Both are located at the centre of the bioreactor chamber 020 behind the bioreactor vessel 100, 200. The collection vessel 033—TFF 034—and TFF pump (not shown) assembly is attached to the background metal sheet 025 of the system 017. Access to collection vessel 033 and TFF 034 is possible when the bioreactor cabinet 001 is not docked into said system 017. The homogeneity inside the collection vessel is guaranteed with the recirculation loop through the TFF 034 with the TFF pump (not shown). From the TFF 034, concentrated biomolecule harvest can be transferred to a downstream chamber 019 of said system. Again, the appropriate pipings 039 are provided to allow fluid transfer. Said downstream chamber 019, flanks said bioreactor chamber 020 on the side opposite from the process chamber 018. The presence of a downstream chamber 019 is optional. In the downstream chamber 019 the harvest can be further clarified after the concentration step in the bioreactor chamber 020. Said downstream chamber 019 is in fluid connection with the bioreactor chamber 020 and comprises one or more purification or filtration devices 032 allowing the purification or filtration of a biomolecule of a cell harvest. The back sheet 025 of the downstream chamber is provided with pumps, pipings, electrical sockets and/or manifolds needed for allowing functioning of said chamber. In the back of the technical enclosure 028 all technical component must be installed like motors, network cables, power supply, etc.

FIG. 6B shows the embodiment of FIG. 6A from a different perspective.

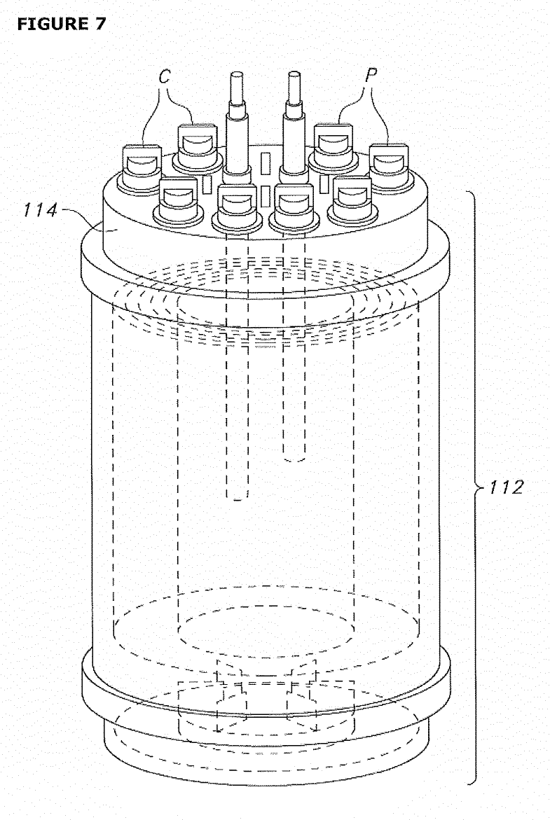

Reference is now made to FIG. 7, which illustrate one embodiment of a bioreactor vessel 100 for culturing cells, according to one aspect of the disclosure. In some embodiments, the bioreactor vessel 100 includes an external casing or housing 112 forming an interior compartment and a removable cover 114 for covering the interior compartment, which may include various openings or ports P with removable covers or caps C for allowing for the selective introduction or removal of fluid, gas (including by way of a sparger), probes, sensors, samplers, or the like.

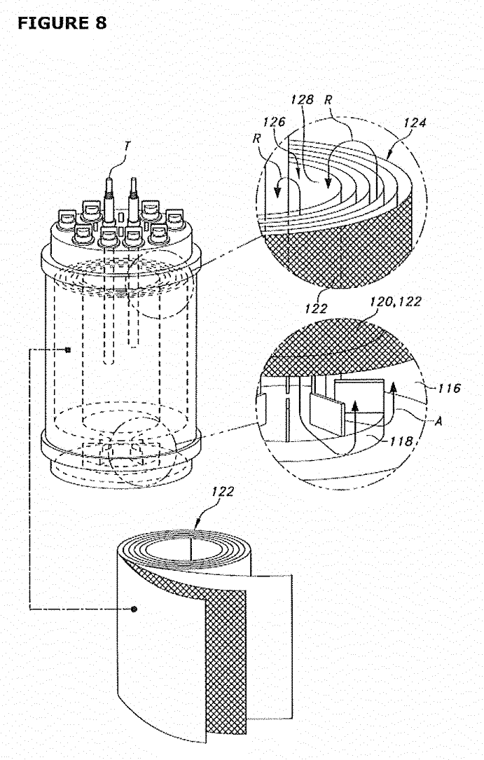

Within the interior compartment formed by the bioreactor housing 112, several compartments or chambers may be provided for transmitting a flow of fluid or gas throughout the bioreactor vessel 100. As indicated in FIG. 8, in some embodiments, the chambers may include a first chamber 116 at or near a base of the bioreactor vessel 100. In some embodiments, the first chamber 116 may include an agitator for causing fluid flow within the bioreactor vessel 100. In some embodiment, the agitator may be in the form of a “drop-in” rotatable, non-contact magnetic impeller 118 (which as outlined further below may be captured or contained within a container (not shown) including a plurality of openings for admitting and releasing fluid).

In some embodiments, as a result of the agitation provided, fluid may then flow upwardly (as indicated by arrows A in FIG. 8) into an annular chamber 120 along the outer or peripheral portion of the bioreactor vessel 100. In some embodiments, the bioreactor vessel is adapted to receive a fixed bed, such as a structured spiral bed 122, which in use may contain and retain cells being grown. As indicated in FIG. 8, in some embodiments, the spiral bed 122 may be in the form of a cartridge that may be dropped or placed into the chamber 120 at the point of use. In some embodiments, the spiral bed 122 can be pre-installed in the chamber during manufacture at a facility prior to shipping.

In some embodiments, fluid exiting the chamber 120 is passed to a chamber 124 on one (upper) side of the bed 122, where the fluid is exposed to a gas (such as oxygen or nitrogen). In some embodiments, fluid may then flow radially inwardly to a central return chamber 126. In some embodiments, the central return chamber can be columnar in nature and may be formed by an imperforate conduit or tube 128 or rather formed by the central opening of the structured spiral bed. In some embodiments, the chamber 126 returns the fluid to the first chamber 116 (return arrow R) for recirculation through the bioreactor vessel 100, such that a continuous loop results (“bottom to top” in this version). In some embodiments, a sensor, for example a temperature probe or sensor T may also be provided for sensing the temperature of the fluid in the chamber 126. In some embodiments, additional sensors (such as, for example, pH, oxygen, dissolved oxygen, temperature) may also be provided at a location before the fluid enters (or re-enters) the chamber 116. The sensors and probes as described herein, may be reusable, one-time-use and/or disposable.

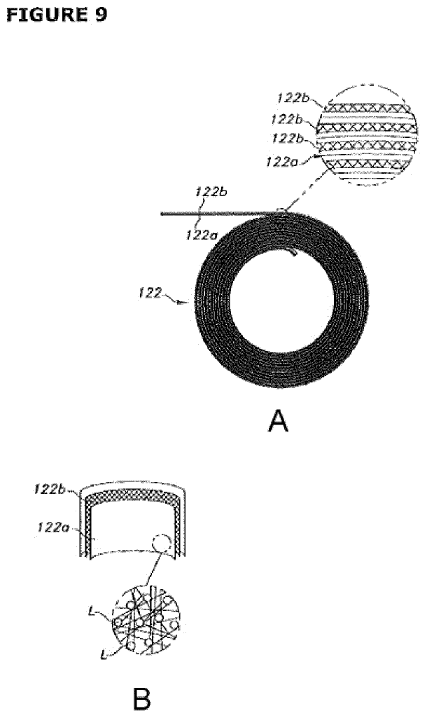

FIG. 9A shows one embodiment of a matrix material for use as a structured fixed bed in the bioreactor vessel of the present disclosure and, in particular, a spiral bed 122. In some embodiments, one or more cell immobilization layers 122a are provided adjacent to one or more spacer layers 122b made from a mesh structure. In some embodiments, the layering may optionally be repeated several times to achieve a stacked or layered configuration. In some embodiments, the mesh structure included in spacer layers 122b forms a tortuous path for cells (see cells L in FIG. 9B suspended or entrapped in the material of the immobilization layer 122a), and a cell culture may form part of any disclosure claimed herein) and fluid to flow when layered between two immobilization layers 122a. Homogeneity of the cells is maintained within the structured fixed bed as a result of this type of arrangement. In some embodiments, other spacer structures can be used which form such tortuous paths. In some embodiments, as shown in FIG. 9A, the structured fixed bed can be subsequently spirally or concentrically rolled along an axis or core (e.g., conduit 128, which may be provided in multiple component parts). In some embodiments, the layers of the structured fixed bed are firmly wound. In some embodiments, the diameter of the core, the length and/or amount of the layers will ultimately define the size of the assembly or matrix. In some embodiments, thickness of each of the layers 122a, 122b may be between 0.1 and 5 mm, 01 and 10 mm, or 0.001 and 15 mm.

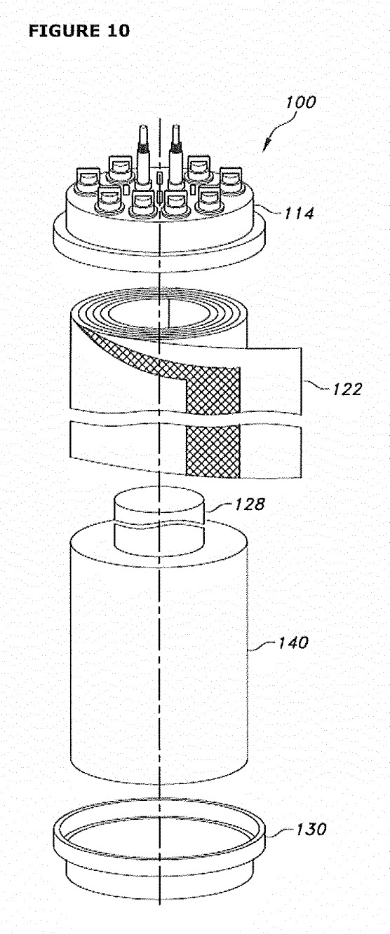

According to one aspect of this disclosure, the bioreactor vessel 100 in certain embodiments may be “modular.” In some embodiments, a modular bioreactor vessel can be comprised of a plurality of discrete modules that interact together to create a space suitable for culturing cells in a manner that is highly predictive due to the manufacturing homogeneity of the modules. In some embodiments, a modular bioreactor vessel is not limited to particular shape or form (e.g., cylindrical or otherwise, and with a structured fixed bed or unstructured bed, depending on the application). For example, as shown in FIG. 10. In some embodiments, the modules may comprise a base portion formed by base module 130, an intermediate portion formed by an intermediate module 140 (which may be formed from a number of stackable modular portions, as outlined further in the description that follows), an optional associated central module, such as conduit or tube 128, which may also be considered part of the intermediate module, and a cover module, such as formed by a cover part in the form of lid or removable cover 114. In some embodiments, the modules may be separately manufactured as individual components and either assembled at a manufacturing facility based on an intended application (and then shipped to a point of use) or assembled based on an intended application at the point of end use. In some embodiments, the modules of the bioreactor vessel 100 interact to create a place for growing cells, such as in a high-density manner using a fixed bed, such as for example a structured or unstructured fixed bed.

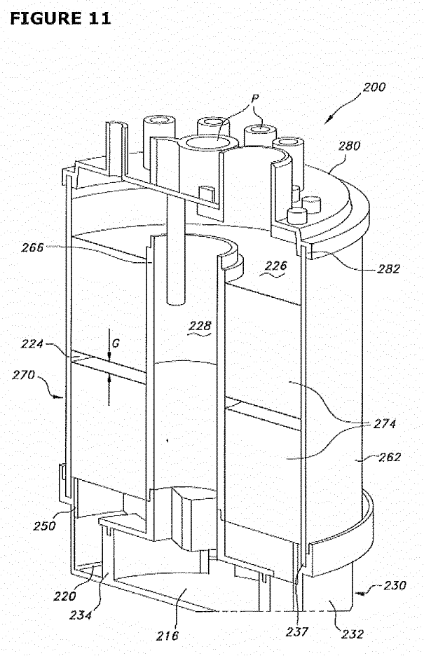

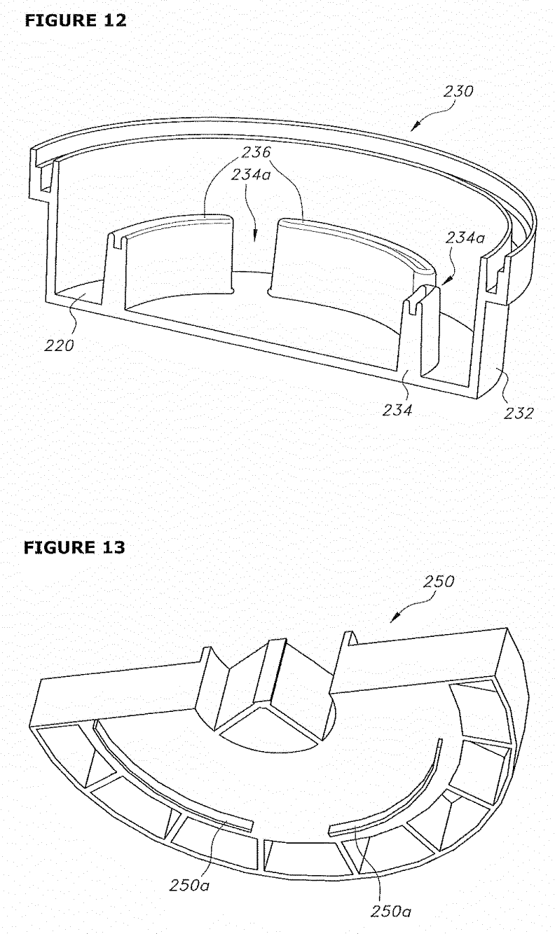

A further embodiment of a bioreactor vessel 200 according to the disclosure is shown in FIGS. 11-14. In some embodiments, the bioreactor vessel (whether modular or otherwise pre-assembled as a single unit) can comprise a base, an intermediate portion and a cover. In some embodiments, a base portion can comprise a base part 230. In some embodiments, an intermediate portion can comprise intermediate parts 250 and/or 270. In some embodiments, intermediate parts 250 and 270 are not identical. In some embodiments, a cover portion can comprise a cover part 280. Referring to FIG. 11, in some embodiments, base part 230 may include an external wall 232 and an internal wall 234, which may define a first chamber 216 for receiving the agitator (not shown). In some embodiments, the internal wall 234 can include openings 234a for allowing fluid flow to the second, radially outward chamber 220 bounded by the external or outer wall 232 (FIG. 12).

As can be seen in FIG. 12, in some embodiments, the internal wall 234 may include a plurality of connectors, such as grooves 236, for engaging corresponding connectors, such as tongues 250a, on the first intermediate part 250, as shown in FIG. 13. In some embodiments, the internal wall 234 may be of lower/higher height than the external wall 232. In some embodiments, the internal wall 234 may be of lower height than the external wall 232, as can be seen in FIG. 8. With reference to FIG. 11, in some embodiments, the first intermediate part 250 may be at least partially recessed within the base part 230.

In some embodiments, the base part 230 may include a peripheral connector, such as a groove 237 (FIG. 11). In some embodiments, the connector or groove 237 can be adapted to receive a corresponding connector of a second intermediate part 270, which may simply be part of an outer wall 262 thereof. In some embodiments, within the intermediate part 270 can be located a plurality of fixed beds 274 in a third chamber 224 (but a single monolithic fixed bed could be used, which in this or any disclosed embodiment may take any size, shape, or form), which could be supported by an interposed support, but a gap G could also be provided between adjacent sections of fixed beds). The gap could also be eliminated, such that an upper bed rests on and is supported by a lower one.

In some embodiments, the structured fixed bed can be of the spiral form, as shown in FIG. 9A (which spiral form can be implemented in any embodiment of a bioreactor vessel, disclosed or otherwise). In the case of a spiral bed, the bed may be wound around an internal wall 266, which may form a fifth chamber 228 for returning fluid to the first chamber 216 in the base part 230. The internal wall 266 may comprise multiple stacked tubular parts, as shown. In some embodiments, the multiple stacked tubular parts can allow for the height to be adjusted depending on the number of fixed beds present (e.g., one tubular part may be provided for each stacked bed) (FIG. 11).

In some embodiments, the cover part 280, or lid can be adapted to removably connect with the second intermediate part 270, and thus form a fourth chamber 226 in which the liquid encounters gas, for example air. In some embodiments, the connection between the cover part and the second intermediate can be by a connector, such as a groove 282, which receives the upper end of the outer wall 262 or any access mechanism disclosed herein. The lid or cover part 280 may include various ports P (FIG. 11).

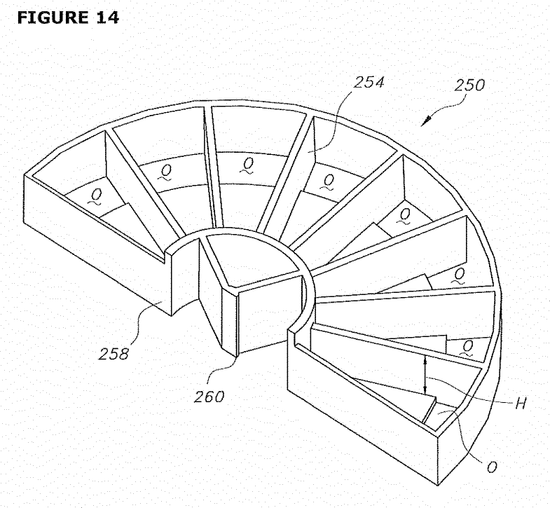

Turning back to FIGS. 11 and 14, further details of the intermediate part 250 are shown. In some embodiments, part 250 may include a plurality of radially extending supports 254, which thus lend support for a structured fixed bed when resting thereon in the adjacent third chamber 224. In some embodiments, the height H of the supports 254 can be sufficient to allow the fluid to develop sufficient upward velocity before entering the chamber 224 to pass through the full section of the fixed bed 274 (FIG. 11).

In some embodiments, an inner annular wall 258 can be connected to the inboard end of the supports 254. In some embodiments, the wall 258, corresponds in diameter to the diameter of the internal wall 266 of the intermediate part 270, which may also connect with it (such as by nesting). In some embodiments, the internal wall 266 can form a passage for delivering fluid from the fifth chamber 228 to the first chamber 216. In some embodiments, a flow disruptor 260 may be provided in this passage to help prevent the creation of any vortex within the fifth chamber 228.

From FIG. 11, in some embodiments, it can be understood that the flow from one fixed bed module to the next-adjacent fixed bed module in the cell culturing chamber 224 can be direct or uninterrupted. In some embodiments, the outer chamber 224 can create a continuous flow path through the multiple beds located therein, which may be structured fixed beds, unstructured fixed beds, or unstructured beds. In some embodiments, the continuous and substantially unimpeded flow through the predesigned and matching bed modules helps to promote homogeneity for cell growth and other processing and enhances the consistency of the cell culturing operation, and also promotes the ability to take measurements or samples from the stacked beds, which is not readily possible if blocking partitions (as contrasted with the perforated supports, as discussed below) are present. Finally, in a structured bed embodiment, the manufacture of the overall bioreactor vessel is even less complicated and labor intensive as the effort to match the properties and characteristics from one fixed bed module to the other is greatly reduced.

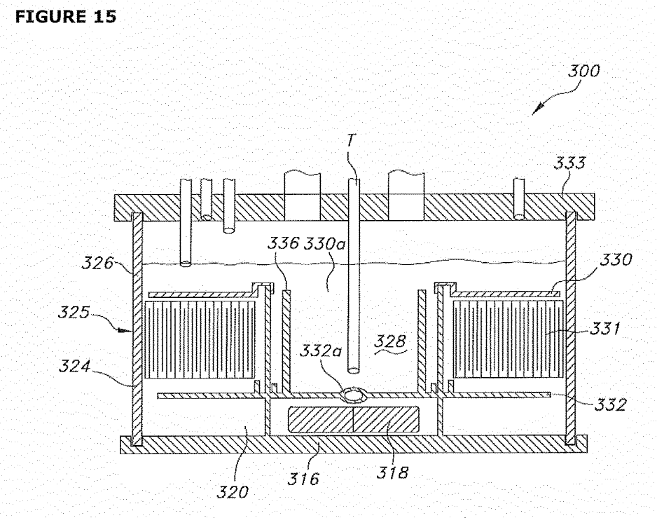

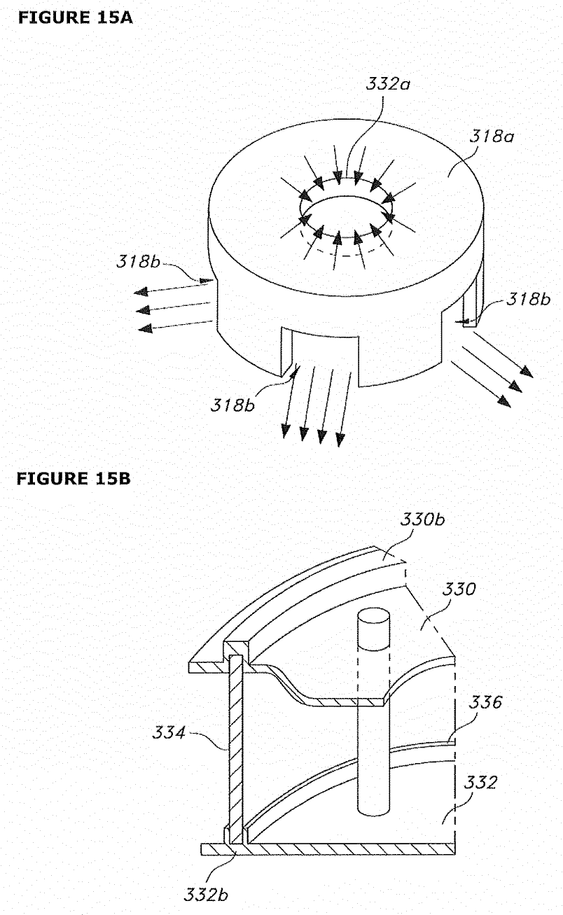

Reference is now made to FIGS. 15 and 16, which schematically illustrate a third embodiment of a bioreactor vessel 300, which for purposes of clarity is shown in cross-section. In some embodiments, the bioreactor vessel 300 (whether modular or otherwise pre-assembled as a single unit) comprises an external housing 331 with a cover 333, either of which may include various openings or ports for allowing for fluid introduction or removal. In some embodiments, within the bioreactor housing 331, several compartments or chambers are provided, including a first chamber 316 including an agitator for causing fluid flow within the bioreactor vessel 300, which may be in the form of a “drop-in” rotatable, non-contact magnetic impeller 318 or an agitator disclosed herein. As indicated in FIG. 15A, in some embodiments, the impeller 318 may be housed, captured or contained within a housing, such as a housing or container 318a including a plurality of openings 318b serving as inlets and outlets for admitting and releasing fluid (but any other form of agitator could be used). In some embodiments, the agitation created may be such that fluid is caused to flow into a second or outboard annular chamber 320, which is radially outward of the first chamber 316.

In some embodiments, fluid may then flow upwardly (as indicated by arrows in FIG. 16) into a third annular chamber 324 along an intermediate, outer portion of the bioreactor vessel 300. In some embodiments, the outer portion can be adapted to receive a fixed bed, such as a structured spiral bed 325, but other forms may be used), which in use may contain cells being grown. In some embodiments, the spiral bed 325 may be in the form of a cartridge that may simply be dropped into the chamber 324 at the point of use, or could be pre-installed in the chamber during manufacture at a facility prior to shipping.

In some embodiments, fluid exiting the third chamber 324 can then passed to a fourth chamber 326, where it is exposed to a gas (such as air) and then flows radially inwardly to a fifth chamber 328, which is columnar in nature and returns the fluid to the first chamber 316 for recirculation through the bioreactor, such that a continuous loop results. In some embodiments, a temperature probe or sensor T, or any other sensor disclosed herein may also be provided for sensing a parameter, for example the temperature of the fluid directly in the fifth chamber, and additional sensors (such as, for example, pH or dissolved oxygen) may also be provided at this location (which is before the fluid enters (or re-enters) the fixed bed 325).

From the partially cutaway image at FIG. 15B, it can be understood that the third chamber 324 may be bounded by upper and lower plates 330, 332, which include openings or perforations for allowing fluid generally free of cells to enter and exit the fixed bed 325. In some embodiments, the lower plate 332 may include a central opening 332a for allowing fluid to pass from the fifth chamber 328 to the first chamber 316 for recirculation. In some embodiments, the upper plate 330 can include an opening 330a, into which fluid may travel to enter the fifth or return chamber 328.

In some embodiments, support for the upper plate 330 may be provided by a hollow, generally cylindrical tube 334, but could take other shapes. In some embodiments, the opposed ends of this tube 334 may fit into corresponding grooves 330b, 332b in the plates 330, 332 (in some cases the lower plate 332 can be integral with the impeller housing or container 318a in the illustrated embodiment). In some embodiments, supports, such as generally vertical rods 336, can be arranged to provide added support for the plate 330. In some embodiments, the disclosed vertical rods 336 do not interfere in any significant way with the fluid flow in the corresponding chamber 328. In some embodiments, the ends of the rods 336 may be recessed in the plates 330, 332, or held in place by suitable fasteners or locking mechanisms (e.g., locking connections, bolts or adhesives).

From FIG. 16 and the action arrows provided thereon, it can be understood that, as a result of the fluid agitation, in some embodiments, fluid may flow from the chamber 316 outwardly into chamber 320. In some embodiments, the fluid can then be redirected to pass vertically through chamber 324 including the fixed bed, and into chamber 328. In some embodiments, fluid is then directed inwardly to chamber 328, where the fluid may return to the first chamber 316 via opening 332a. In some embodiments, fluid can refer to culture medium.

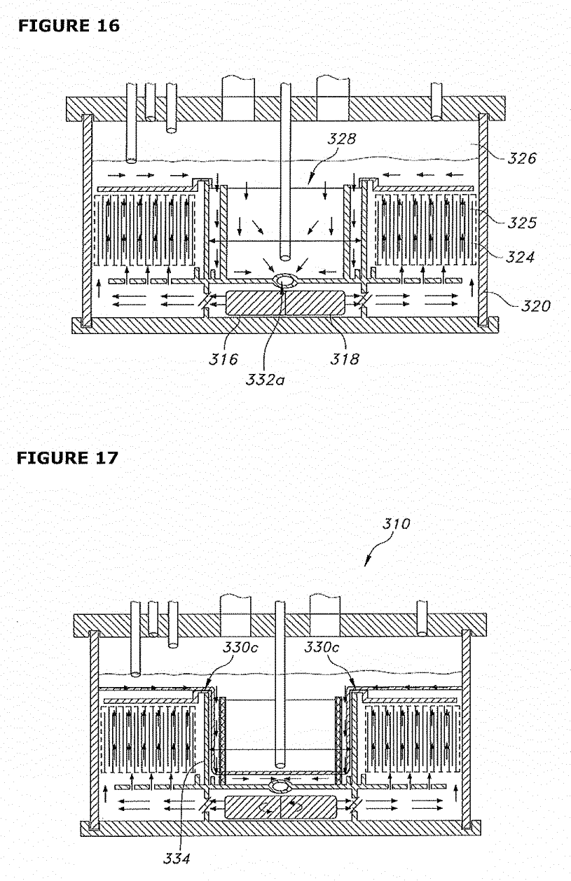

FIG. 17 further illustrates an arrangement in which, in some embodiments, the upper plate 330 is provided with peripheral openings 330c to allow fluid to flow directly along the inner wall formed by tube 334. In this manner, a thin layer or film of fluid may be created, which flows downwardly while passing through the fifth chamber 328. In some embodiments, this may serve to increase the volume of the fluid exposed to gas (air) within the fifth chamber 328, prior to it being returned to the first chamber 316. In some embodiments, this implementation can allow for more oxygen transfer which may be needed for larger sizes or otherwise to increase cell growth rates adjust process parameters based on the biologic being produced. In some embodiments, the “waterfall” implementation that creates a fluid film can be achieved by adding a limited quantity of cell culture medium from the start, such that only a small overflow results. Alternatively, in some embodiments, the “waterfall” implementation is achieved by adding cell culture medium and cells and then when cells are growing in the bed, withdraw culture medium (such as using a dip tube) in the corresponding chamber, such as chamber 328.

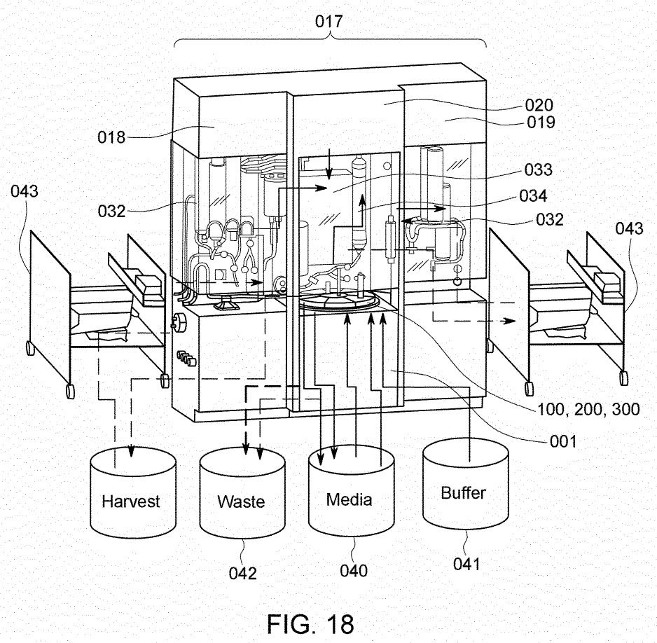

FIG. 18 illustrates a possible process flow in an embodiment of the system 017. Said process involves the production of a biomolecule, such as a viral particle, e.g. for producing a vaccine or a viral gene therapy product. To this purpose cells are cultured in the bioreactor vessel 100,200,300 inside the bioreactor cabinet 001 which is embedded in the bioreactor chamber 020. Media 040 and buffer 041 are supplied to the bioreactor vessel by means of externally supplied bags, that are connected to the bioreactor chamber. Waste that is produced during the production cycle is guided towards a waste vessel 042. Subsequently the bioreactor harvest is lysed and transported to the process chamber 018, where it is filtered using purification or filtration devices 032. After this step, the product is either harvested or transported to the bioreactor chamber 020, where it is concentrated by means of the collection vessel 033 and TFF 034. Afterwards, the concentrate is transported towards the purification or filtration devices 032 in the downstream chamber 019. Additional chambers 043 can be connected to said system in case further upstream or downstream processing is needed.

It will be apparent to the skilled person that the process flow as shown in FIG. 18 is exemplary and that other sequences of process flows may be used in relation to the currently disclosure.

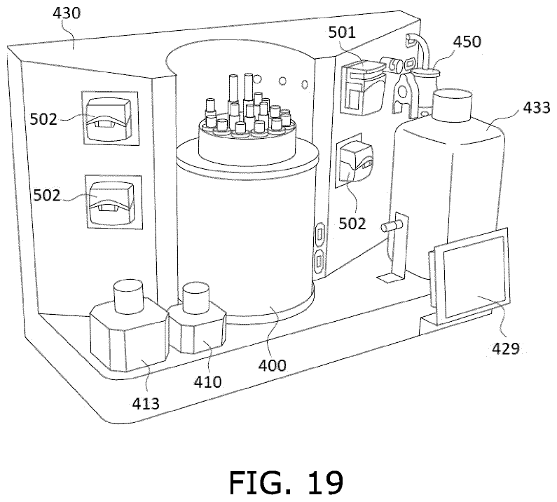

FIG. 19 shows an embodiment of the system of the current disclosure. FIG. 19 shows a system designed to be used in a biosafety cabinet or isolator and can be used for both process development work and pilot-scale production of biological material, in which case it can be used to produce material for clinical trials as well as low volume commercial production. The system is designed to be used for the growth of adherent cells, as well as non-adherent cells. To that purpose, the system comprises a bioreactor vessel 400, preferably a fixed bed bioreactor. The fixed bed of the bioreactor vessel can be provided with structural elements for allowing growth of the cells on the surface of said elements. The elements can be made of polyethylene, preferably hydrophilized polyethylene. In an embodiment the bioreactor vessel 400 is for single-use only. Conduits present in the system for liquid or gas transport are not shown in the figure. The bioreactor vessel 400 has at least two fluid connections, wherein one connection allows entrance of fluid into the bioreactor vessel and a second connection allows removal of fluid. This last connection is designed in such way that it minimizes dead space inside the bioreactor vessel 400 once emptied. In a further embodiment, said bioreactor vessel 400 is provided with gas connections, for allowing entrance and/or exit of gas. In a preferred embodiment, three gas connections are present, two connections entering the bioreactor vessel 400 and one connection exiting said bioreactor vessel 400. Advantageously, the bioreactor vessel 400 is furthermore designed to allow sampling for both in-process control and for end of process analysis, preferably from the top of said bioreactor vessel 400. Sampling can occur via syringes or equivalent assemblies.

Circulation in the bioreactor vessel 400 is achieved by use of an impeller, preferably a magnetically driven impeller. A heating element may be present to heat the content of said bioreactor vessel 400, or to heat medium that is brought into said bioreactor vessel 400. The lid of the bioreactor vessel 400 is provided with one or more sensors for measuring temperature, pH and/or dissolved oxygen in said bioreactor vessel 400.

Liquid output from the bioreactor vessel 400 will be transferred by means of a conduit to a collection vessel 433 also known as concentrator bottle. Such collection vessel 433 may be a PET bottle, and may hold a volume of about 500 m¹ to 5000 mL. This collection vessel 433 is connected to a concentrator 450 which may be a TFF. Liquid from the collection vessel 433 comprising the target biomolecule will be transported to the concentrator 450 by means of a pump 501. Said pump 501 is, in an embodiment, able to provide a shear rate of 2000 s−1 inside the concentrator 450. The retentate of the concentrator 450 will subsequently be brought back to the collection vessel 433, whereas liquid waste will be discarded (preferably to a waste bottle, not shown on FIG. 19). Due to the re-circulation of retentate back and forth from the collection vessel 433 to the concentrator 450, a heavily concentrated biomolecule product will be obtained, which can be used for further downstream processing (such as chromatographic purification) or as source for trials such as e.g. clinical trials.

The process flow from bioreactor vessel 400 to concentrator 450 is controlled by a process controller. In order to maintain the compactness of the system, especially considering it is sized to be used inside a biosafety cabinet or isolator, the controller is integrated in a docking station 430 which is designed to receive the above-described bioreactor vessel 400, concentrator 450 and collection vessel 433. The controller controls and operates bioreactor vessel parameters as well as process flow parameters and monitors and records data from one or more sensors described above (pH, temperature and/or DO). Said controller further controls the functioning of the concentrator 450 and the recirculation of retentate from concentrator 450 to collection vessel 433 and back, preferably by controlling the functioning of the pump(s) 501, 502 between collection vessel 433 and concentrator 450. A first and a second pressure sensor (not shown) allow for determining the volume of liquid in the bioreactor vessel 400.

To that purpose, said controller is provided with software allowing monitoring, controlling and recording the process flow and parameters of the system. Access to the controller can be provided to the user via a computer which is pluggable to the controller. The controller allows export of data through one or more USB connections present on said docking station and allows access to an IT network. A screen 429 such as a touch screen present on the docking station allows the user to follow the process flow and measured parameters as well as to manually operate the system, e.g. by starting or stopping certain sub-processes.

As described above, the docking station 430 with integrated controller further allows for docking of a bottle for supply of base 413 to the bioreactor vessel 400. Such bottle may be a PET bottle, with a volume of between 500 mL to 5000 ml. Said docking station 430 may further allow docking of a bottle for supply of inoculum 410/additive (not shown) to the bioreactor vessel 400. A retention tray for catching potential liquid overflows can be provided.

The docking station 430 will be preferably constructed out of a material that allows cleaning with a NaOH (such as 0.5 M NaOH) solution, alcohols such as ethanol or virucides such as Virkon. The docking station 430 should equally be able to resist a sterilizing regime using vaporized hydrogen peroxide (VHP). In a preferred embodiment, the material of said docking station 430 is a corrosion resistant metal. The docking station 430 can be powered by a power supply, such as a standard 110-230V, 50-60 Hz power supply.

FIG. 20 shows an embodiment of the system of the current disclosure depicting conductors for detecting the presence of foam in a vessel. The conductors 150 formed by pin 152 and additional conductors 150, such as pins 148a, 148b, may extend through a port 114a in the lid 114, but could extend through any other portion of the vessel 110. The difference of detectable electrical signal (e.g., potential, impedance, capacitance) between the conductors 150 formed by pin 152 and additional conductors 150, such as pins 148a, 148b, at different heights would not be the same when contacted by liquid (L) or foam. This potential difference allows the measurement of several levels in combination with a grounding conductor 150. In such case, one skilled in the art can appreciate that the lowest conductor (148a) must remain submerged in order for this arrangement to work. As the levels of liquid or foam increase or decrease, the difference may be detected by the different levels in contact with the conductors 150 and thus provide an indication of the level of liquid or foam. Besides the detection of foam, a first and a second pressure sensor (not shown) allow for determining the volume of liquid in the vessel.

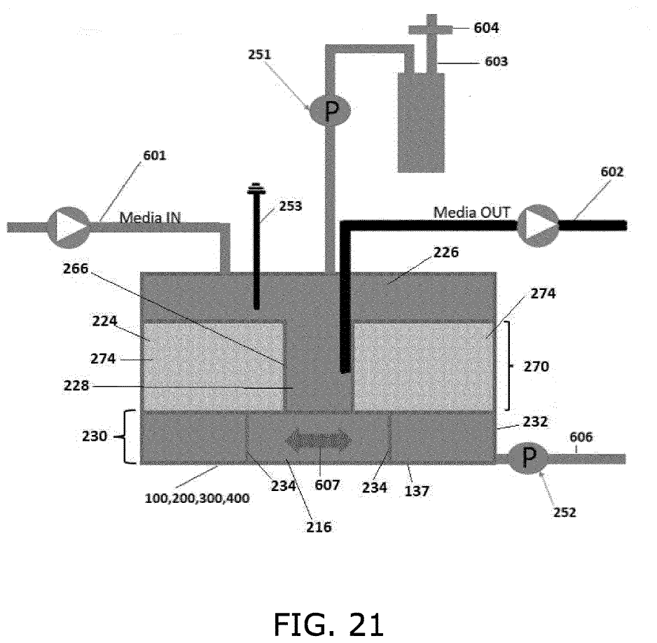

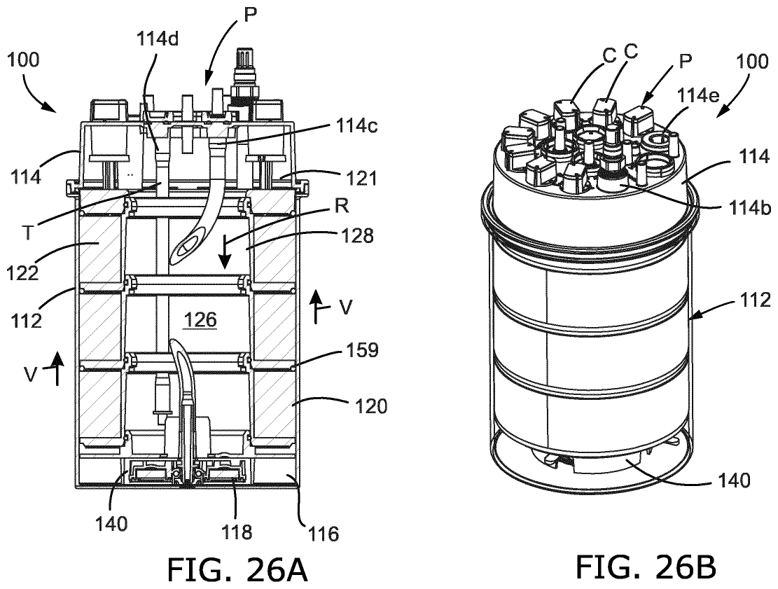

FIG. 21 shows an embodiment of the system according to the current disclosure depicting a bioreactor vessel and associated pressure sensors for determining the volume or weight of liquid in said vessel. The biomolecule production system comprises a bioreactor vessel 100,200,300,400 for cultivation of cells or organisms for biomass expansion and/or production of a biological product or biomolecule (not shown). The bioreactor vessel comprises a base, an intermediate portion and a cover. The base portion comprises a base part 230. The intermediate portion comprises an intermediate part 270. Base part 230 includes an external wall 232 and an internal wall 234, which define a first chamber 216 for receiving an agitator 607. The intermediate part 270 comprises one or more fixed beds 274 in a third chamber 224. The fixed bed is wound around an internal wall 266, which forms a fifth chamber 228 for returning fluid to the first chamber 216 in the base part 230. In the fourth chamber 226 the liquid encounters gas, for example air. The level of culture media (not shown) inside the bioreactor vessel 100,200,300,400 is controlled by providing media through a media inlet line 601 and by pumping media out by means of a media outlet line 602 and a pumping mechanism (not shown). Determination of the liquid level in said bioreactor vessel 100,200,300,400 is important. As such, the bioreactor vessel 100,200,300,400 is equipped with a first 252 and a second 251 pressure sensor for determining the volume or weight of liquid in said bioreactor vessel 100,200,300,400. The first pressure sensor 252 in positioned in the drain line 606 of the bioreactor vessel 100,200,300,400 and measures the hydrostatic pressure in the bioreactor vessel 100,200,300,400. The positioning of the first pressure sensor 252 is important and should be as close as possible to the bottom wall 137 of the bioreactor vessel 100,200,300,400. As the headspace of the bioreactor vessel 100,200,300,400 is not at atmospheric pressure, a second pressure sensor 251 is required to measure the gas headspace pressure. The second pressure sensor 251 is incorporated in a two flanged design which is connected by means of tri-clamp gaskets (not shown) and clamps (not shown) to the bioharvest vessel 100,200,300,400 and to an outlet gas line 603 protected by a vent filter 604. The total liquid volume in the bioreactor vessel 100,200,300,400 consists of a first volume of liquid below the first pressure sensor 252 and a second volume of liquid above the first pressure sensor 252, said total liquid volume is determined by: calculating the first volume of liquid and adding this to the second volume of liquid, said second volume of liquid being determined by measuring the hydrostatic pressure by means of the first pressure sensor 252, measuring the gas headspace pressure by means of the second pressure sensor 251 in the headspace of the bioreactor vessel 100,200,300,400, thereby determining the differential pressure and calculating the volume of fluid above the first pressure sensor 252. The sensors 251,252 are connected to monitors or control systems, such as a process controller (not shown). In addition, a stainless steel pin 253 is placed in the bioreactor vessel 100,200,300,400 for the detection of foam. By adequately measuring the level of liquid in the bioreactor vessel 100,200,300,400, the pump for pumping media out of the bioreactor vessel 100,200,300,400 can be controlled based on the volume of liquid in the bioreactor vessel 100,200,300,400 without sucking the media at the air-liquid interface and thus preventing the presence of gas bubbles in the media outlet line 602 (and further downstream processing equipment). In addition, when the level of fluid in the bioreactor vessel 100,200,300,400 is too low, a warning signal could be triggered. Likewise, a similar system comprising a first 252 and a second 251 pressure sensor can be used to determine the volume or weight of liquid in a bioreactor vessel 100 according to FIGS. 26A-26B. The bioreactor or bioreactor vessel 100 of FIGS. 26A-B includes an external casing or housing 112. The housing 112 forms an interior compartment in which cell culturing may be completed using various components or techniques. According to one aspect of the disclosure, the housing 112 may in some embodiments form a vessel comprising a single-piece or monolithic structure, such as a pot or bucket having an open top. Providing such a vessel may eliminate the cost and complexity of forming the housing 112 from multiple parts fixed together, such as using welding or adhesives. Furthermore, such a construction avoids the need for associated hermetic seals in the body of the housing 112, thus eliminating the possibility of leakage and/or contamination, and improves bioreactor integrity. Fabrication of this single piece housing 112 may involve using injection-molding techniques, 3D printing, or other methods such that no seams exist in order to minimize exposure to contamination. In some applications, the housing 112 may be translucent or transparent. In other applications, the housing 112 may be opaque, and may be made of any material, but a preference for plastics exists to allow for a single-use arrangement, if desired. A cover or lid 114 may overlie the open top of the housing 112 to cover or seal the interior compartment thereof. In one embodiment, the lid 114 is designed to be easily removable, such as by being secured in place by an interlocking engagement with the housing 112 (including possibly a friction-fit or bayonet fitting), but removable fasteners could also be used, such as tabs and/or clips which may interlock with one another, clamps, and/or screws. This facilitates opening the bioreactor 100 and may avoid the need for using samplers (which tend to increase cost and may be challenging to implement in particularly small vessels in view of the size constraints). Together, the housing 112 and the lid 114 may comprise a container for containing the remaining elements of the bioreactor. The lid 114 may include various openings or ports P with removable closures or caps C for allowing for the selective introduction or removal of material, fluid, gas, probes, sensors, samplers, or the like, and lends flexibility to the design. In particular, the lid 114 may include holders 114b, such as for receiving suitable sensors (e.g., temperature, capacitance, permittivity, biomass, metabolite such as glucose or lactate, pressure, flow measurement, fluid level, pH or DO probes, or the like). As best shown in FIG. 26A, an internal connector 114c for a conduit or tubing form part of the lid 114. The lid 114 may further include a corresponding connector 114d for a media extraction tube T. As shown in FIG. 26B, removable caps 114e with suitable seals, such as O-rings, may permit auxiliary access, if needed. Sampling ports for receiving samplers, such as in the form of probes, may also optionally be provided in the lid 114. Within the interior compartment formed by the housing 112, several compartments or chambers receive and transmit a flow of fluid, gas, or both, throughout the bioreactor 100. As indicated in FIG. 26A, the chambers may include a first chamber 116 at or near a base of the bioreactor 100. In some embodiments, this first chamber 116 may include an agitator for causing fluid flow within the bioreactor 100. In some embodiments, the agitator may be in the form of a “drop-in” rotatable, non-contact magnetic impeller 118, which thus forms a centrifugal pump in the bioreactor 100. Instead of such an impeller 118, the agitator could also be in the form of a stir bar, an external pump forming part of a fluid circulation system, or any other device for causing fluid circulation within the bioreactor. The agitation provided results in fluid flowing upwardly (as indicated by arrows V in FIG. 26A) into a second chamber, which may be a peripheral chamber 120 formed in and extending along the outer or peripheral portion of the bioreactor 100. Alternatively, the bioreactor 100 could be adapted to allow fluid to flow in an opposite direction. In some embodiments, the bioreactor 100 is adapted to house a cell culture bed 122 in any form including a packed bed, fixed bed, a structured fixed bed, a fluidized bed, etc., Fluid exiting the second, peripheral chamber 120 is passed to a headspace formed by an upper chamber 121 on one (upper) side of the bed 122, where the fluid is exposed to a gas (such as oxygen). Fluid may then flow radially inwardly to a third, central chamber 126 to return to the lower portion of bed 122. In some embodiments, this central chamber 126 can be columnar in nature, formed by one or more imperforate conduits or tubes 128 (which may comprise multiple annular portions of fixed bed supports, each including a portion of the fixed bed, as outlined further below), and the flow may be such that a waterfall-like arrangement is created. The central chamber 126 returns the fluid falling or otherwise entering it to the first, base chamber 116 (arrow R showing return path) for recirculation through the bioreactor 100, such that a continuous loop results (“bottom to top” in this version, but such could be reversed or otherwise modified without departing from the disclosure). The bioreactor 100 may include a support for supporting the fixed bed. In one form, this support may comprise a container 140 for containing the agitator, such as impeller 118, in an interior compartment of the housing 112. The container 140 may be adapted to receive fluid from a central opening and eject the fluid radially outwardly via one or more openings (e.g., four spaced 90 degrees apart), such as a result of the movement (rotation) of the agitator, such as impeller 118. The container 140 may further include one or more outward projections, which serve as positioners for centering or uniformly spacing the container from an inner wall of the housing 112, but without being attached to it. For example, the container 140 along an upper portion may include one or more radially extending arms. These arms may be adapted for aligning or centering the container within the housing 112 of the bioreactor when rested on a surface thereof, such as the floor. While the arms may be on the container 140, the arms may instead attach to the inner wall of the housing 112 and extend toward the container, but not attach to it, to facilitate easy removal.

FIG. 22 shows a schematic overview of a system for producing biomolecules according to an embodiment of the disclosure. The schematic overview is shown of a system for producing biomolecules comprising a bioreactor (400) including a chamber suitable for receiving a liquid comprising cells and viral particles, a concentrator (450) and a collection vessel or concentrator bottle (433). Multiple types of concentrators are suitable for use in the system, the system according to this embodiment, is provided with a tangential flow filtration device (TFF) acting as the concentrator. The concentrator is equipped with a retentate line output (303) which collects the concentrator output and which allows re-circulating of the retentate output to an input of the collection vessel (433). Two gas connections are present, one connection (304) entering the bioreactor (400) and one connection (305) exiting said bioreactor (400). The bioreactor (400) is further connected with an inoculum vessel and vessels containing culture media to feed the cells during the growth, infection, transfection and production process. Additional vessels could be connected to optionally lyse the cells at the end of the process and/or rinse the bioreactor. The system conduits are fitted with pumps (501, 504-506) and valves (601) to provide directional liquid flow, to control differential pressure between different fragments of the system and to provide cross-flow of the liquid through the TFF concentrator (450). The bioreactor (400) and the collection vessel (433) are connected by a conduit having a feeding pump (504), facilitating liquid transport from the bioreactor (400) to said collection vessel (433). Alternatively, an additional conduit connected directly from the bioreactor (400) to the concentrator (450) could be present (not shown on figures) for transporting liquid from the bioreactor (400) to the concentrator (450). In addition, the collection vessel (433) and the concentrator (450) are also connected by a conduit (306) having pump (501) which facilitates liquid transport from the collection vessel (433) to the concentrator (450). The concentrator (450) is able to enhance the amount of target biomolecule present in the liquid by enabling the reduction of the total liquid volume without reducing the amount of target molecule in the liquid. During such a concentration step, the permeate from the concentrator (450) is transported towards a waste vessel (308) by means of a permeate conduit (307). Furthermore, the retentate line output (303) which collects the concentrator output and which allows re-circulating of the retentate output to an input of a collection vessel (433) is provided with a pressure control valve (PCV, 601) which allows to maintain a specific transmembrane pressure (TMP) setpoint in the system.

During production of biomolecules, accurate determination of the volume (or weight), and hence the level of fluid, inside one or more of the vessels (such as the bioreactor or collection vessel) of the biomolecule production system is necessary. In the current invention, the collection vessel (433) is equipped with a first (52) and a second pressure sensor (51) for determining the volume and/or weight of liquid in said collection vessel (433), said first pressure sensor (52) measures the hydrostatic pressure in the collection vessel (433) and said second pressure sensor (51) measures the gas headspace pressure in said collection vessel (433). The first and second pressure sensor allow to determine the volume (or the weight) of liquid in said vessel. The ability to determine the liquid level from the pressure in the vessel is based on Pascal's Principle as described above.

The TFF is equipped so that it retains practically all of the target biomolecules in the retentate, while permitting smaller contaminants such as growth medium and solutes to pass through the pores of the membrane and end up in the permeate. The TFF concentrator (450) mediates re-circulating of the retentate comprising the target biomolecule to an input of the collection vessel (433). An output conduit (307) line from the TFF concentrator (450) to a decontamination vessel (308) is provided to discard the permeate. As such, concentration of the liquid in the system can be obtained by the concentrator (450). However, when the output conduit line (307, permeate line) is closed, no permeate leaves the system, and the overall volume is simply recirculated through the concentrator (450) back to the collection vessel (433). The liquid flow from the bioreactor (400) to the collection vessel (433) is controlled by means of a pump (504), which allows harvest feeding from the bioreactor (400) to the collection vessel (433). When the output conduit line (307, permeate line) is closed, the volume of liquid in the collection vessel (433) increases because of the harvest feeding from the bioreactor (400) to the collection vessel (433).

Determination of the liquid level in the vessels of the biomolecule production system is important, for instance to characterize the content inside said vessels, to prevent overfilling of said vessels or to maintain a constant volume in said vessels. This is especially true for systems operating with a perfusion bioreactor (400) (where culture medium is continuously exchanged: fresh medium replenishes nutrients and carbon sources, while cellular waste and medium depleted of nutrients are removed) and where a collection vessel (433) and concentrator (450) are present. The pressure sensors (51,52) are used to determine the liquid level inside the vessel which can be used to characterize the content inside said vessel. For instance, the liquid level inside the vessel can be used to characterize the concentration of target biomolecules inside the collection vessel in the final cell culture harvest after concentration by the concentrator (450) and to determine when concentration of the harvest is sufficient and can be halted. Determination of the liquid level in the vessels of the biomolecule production system further allows to prevent overfilling in said vessels. Likewise, in order to maintain a constant concentration level in the collection vessel (433) throughout diafiltration and/or clarification, the addition of the buffer is metered by adjusting the flow rate of the buffer pump (not shown) based on the weight (and hence level) measurement as determined by means of the pressure sensors.

The process flow in the system (from bioreactor (400) to concentrator (450) and/or collection vessel (433) and between the concentrator (450) and the collection vessel (433)) is controlled by a process controller.

The concentrator (450) follows a cycle to maximize the yield of target biomolecules, wherein either the liquid is simply recirculated through the concentrator (450) (referred to as “recirculation strategy”) or wherein the liquid is concentrated (referred to as “concentration strategy”) depending on the volume (or the weight) in the collection vessel (433) (see FIGS. 23 and 24A-B) as determined by means of the pressure sensors (51,52). The chosen strategy is determined by certain threshold values (see FIG. 23). For instance, “Threshold 1” is the weight (or volume) to start the recirculation strategy (see FIG. 24A), during which the output conduit line (307, permeate line) is closed by means of valve 600, no permeate leaves the system and the volume and weight in the collection vessel (433) increases by the harvest feeding from the bioreactor (400) to the collection vessel (433). This weight increase can be determined by means of the first (52) and second (51) pressure sensor. At a certain point, “Threshold 3” is reached, indicating a high level in the collection vessel (433) as measured by the pressure sensors (51,52), allowing the valve (600) controlling the output conduit line (307, permeate line) to open and start the concentration strategy during which the permeate leaves the system and the retentate comprising the target biomolecule is recirculated to an input of the collection vessel (433) (see FIG. 24B). “Threshold 2” is the final weight desired by the user at the end of the cycle. The end of harvest feeding automatically triggers the concentration until “Threshold 2”. These threshold values are configurable by the user.

During the recirculation strategy the permeate line (307) is closed by a valve (600) and simple recirculation through the TFF cartridge (450) occurs while the PCV valve (601) is 100% open. During the concentration strategy the permeate line (307) is open, allowing the permeate to leave the system, while opening of the PCV valve (601) is done to maintain a specific TMP setpoint in the system.

Furthermore, based on the weight in the collection vessel (433) as determined by means of the pressure sensors (51,52), the flow of the feeding pump (504) during in-line perfusion and concentration is controlled to avoid overfilling (where the maximum level is for instance defined by threshold 3) (see FIGS. 23 and 25A). Similarly, besides monitoring and controlling the liquid level in the collection vessel (433) during harvest feeding from the bioreactor (400), it is also important to determine and control the liquid level in the vessels of the biomolecule production system during diafiltration and clarification. During constant-volume diafiltration and clarification, buffer is introduced into the collection vessel (433) at the same rate that permeate is removed from the system. In order to keep the total volume of retentate constant (and to maintain a constant concentration level in the collection vessel (433)) throughout the process, the addition of buffer is metered by adjusting the flow rate of the buffer pump (not shown) based on the weight (and hence level) measurement as determined by means of the pressure sensors. Likewise, at the end of the harvest cycle the collection vessel (433) needs to be drained. Based on the weight in the collection vessel (433) (as determined by means of the pressure sensors (51,52)) the end of the draining step of the collection vessel (433) can be determined and the flow of the pump (501) transporting liquid to the concentrator can be controlled to automatically stop the draining (and for instance prevent air from entering the filters) (see FIG. 25B). Due to the re-circulation of retentate back and forth from the collection vessel (433) to the concentrator (450), a heavily concentrated biomolecule product will be obtained, which can be used for further downstream processing (such as chromatographic purification) or as source for trials such as e.g. clinical trials.

The present disclosure is in no way limited to the embodiments described in the examples and/or shown in the figures.

1-41. (canceled)

42. A biomolecule production system for producing biomolecules, comprising: one or more vessels selected from the group consisting of a bioreactor vessel, a transfer vessel, a reagent vessel, a waste vessel, a collection vessel, and a concentrator, wherein at least one vessel of said one or more vessels includes at least a first pressure sensor and a second pressure sensor, said first pressure sensor measuring hydrostatic pressure in the at least one vessel and said second pressure sensor measuring gas headspace pressure in said at least one vessel.

43. The biomolecule production system according to claim 42, wherein said first pressure sensor is located in or near the lower half of the at least one vessel.

44. The biomolecule production system according to claim 43, wherein said first pressure sensor is located at a height equal or less than ¼^{th} of a total length of said at least one vessel, which height is measured from a bottom wall of said at least one vessel.

45. The biomolecule production system according to claim 42, wherein said second pressure sensor is located in or near an upper half of the at least one vessel.

46. The biomolecule production system according to claim 45, wherein said second pressure sensor is located at a height equal or greater than ¾^{th} of a total length of said at least one vessel, which height is measured from a bottom wall of said at least one vessel.

47. The biomolecule production system according to claim 42, wherein said at least one vessel includes a drain line, said drain line including the first pressure sensor.

48. The biomolecule production system according to claim 42, wherein said first pressure sensor and said second pressure sensor are removably connected to said at least one vessel.

49. The biomolecule production system according to claim 42, wherein said first pressure sensor or said second pressure sensor is connected to an outside wall of the at least one vessel.

50. The biomolecule production system according to claim 42, wherein said first pressure sensor or said second pressure sensor is indirectly connected to the at least one vessel by tubing.

51. The biomolecule production system according to claim 42, wherein said at least one vessel has an internal volume of at most 100 liters.

52. The biomolecule production system according to claim 42, said system comprising a bioreactor vessel, a concentrator and a collection vessel, wherein said bioreactor vessel and said collection vessel are connected by a first conduit, facilitating liquid transport from said bioreactor vessel to said collection vessel and wherein said collection vessel and said concentrator are connected by a second conduit facilitating liquid transport from said collection vessel to said concentrator.

53. The biomolecule production system according to claim 52, wherein said bioreactor vessel comprises a structured fixed bed for culturing cells.

54. A method for producing a biomolecule comprising the steps of: providing a biomolecule production system comprising a bioreactor vessel, a collection vessel, a concentrator and a waste vessel, collecting a harvest from said bioreactor vessel in the collection vessel and further concentrating said harvest using the concentrator, wherein at least one of said vessels is equipped with at least a first pressure sensor and a second pressure sensor, said first pressure sensor measuring hydrostatic pressure in the at least one vessel and said second pressure sensor measuring gas headspace pressure in said at least one vessel; and calculating a volume or weight of liquid in said at least one vessel based on the measurement of hydrostatic pressure and gas headspace pressure.

55. The method according to claim 54, further comprising controlling liquid flow between said bioreactor vessel, said collection vessel, said concentrator or said waste vessel based on the measurement of hydrostatic pressure and gas headspace pressure in the at least one vessel, by way of pumps and valves and wherein
the functioning of the pumps and valves is automatically controlled by a process controller coupled to said first pressure sensor and said second pressure sensor.

56. The method according to claim 55, wherein said process controller automatically prevents overfilling of said at least one vessel or
automatically maintains a constant volume or a constant concentration of biomolecules in said at least one vessel during one or more diafiltration or clarification steps of the harvest from the bioreactor vessel or
automatically starts or halts a concentration step of the bioreactor vessel harvest by controlling the pumps and valves controlling liquid flow between the collection vessel, the concentrator and the waste vessel, thereby obtaining a predetermined biomolecule concentration or
automatically starts or halts draining of the at least one vessel.

57. A method for determining a total liquid volume in a vessel of a biomolecule production system using a process controller, said vessel comprising at least a first pressure sensor and a second pressure sensor coupled to said process controller, wherein said total liquid volume comprises a first volume of liquid below the first pressure sensor and a second volume of liquid above the first pressure sensor, said total liquid volume is determined by: calculating the first volume of liquid, adding the calculated first volume of liquid to the second volume of liquid, said second volume of liquid being determined by measuring hydrostatic pressure using the first pressure sensor, measuring gas headspace pressure by means of the second pressure sensor, thereby determining a differential pressure, and calculating the second volume of liquid above the first pressure sensor, wherein the differential pressure is comprised in a range between 0 to 200 mbar.
